SEQUENCE LISTING (21 sequences)

SEQ ID NO: 1 - DNA, 27 bp, Artificial Sequence
  Forward primer for PCR
caaggatccgcgcccatcacggcgtac

SEQ ID NO: 2 - DNA, 37 bp, Artificial Sequence
  Reverse primer for PCR
gggtgatctcattttgaacagcgcccagtctgtatag

SEQ ID NO: 3 - DNA, 37 bp, Artificial Sequence
  Forward primer for PCR
ctatacagactgggcgctgttcaaaatgagatcaccc

SEQ ID NO: 4 - DNA, 30 bp, Artificial Sequence
  Reverse primer for PCR
ctgctcgagttagcactcttccatttcatc

SEQ ID NO: 5 - DNA, 2073 bp, Hepatitis C Virus
ggatccgcgcccatcacggcgtacgcccagcagacaaggggcctcctagggtgcataatcaccagcctaactggccgggacaaaaaccaagtggagggtgaggtccagattgtgtcaactgctgcccaaaccttcctggcaacgtgcatcatcaatggggtgtgctggactgtctaccacggggccggaacgaggaccatcgcgtcacccaagggtcctgtcatccagatgtataccaatgtagaccaagaccttgtgggctggcccgcttcgcaaggtacccgctcattgacaccctgcacttgcggctcctcggacctttacctggtcacgaggcacgccgatgtcattcccgtgcgccggcggggtgatagcaggggcagcctgctgtcgccccggcccatttcctacttgaaaggctcctcggggggtccgctgttgtgccccgcggggcacgccgtgggcatatttagggccgcggtgtgcacccgtggagtggctaaggcggtggactttatccctgtggagaacctagagacaaccatgaggtccccggtgttcacggataactcctctccaccagtagtgccccagagcttccaggtggctcacctccatgctcccacaggcagcggcaaaagcaccaaggtcccggctgcatatgcagctcagggctataaggtgctagtactcaacccctctgttgctgcaacactgggctttggtgcttacatgtccaaggctcatgggatcgatcctaacatcaggaccggggtgagaacaattaccactggcagccccatcacgtactccacctacggcaagttccttgccgacggcgggtgctcggggggcgcttatgacataataatttgtgacgagtgccactccacggatgccacatccatcttgggcattggcactgtccttgaccaagcagagactgcgggggcgagactggttgtgctcgccaccgccacccctccgggctccgtcactgtgccccatcccaacatcgaggaggttgctctgtccaccaccggagagatccctttttacggcaaggctatccccctcgaagtaatcaagggggggagacatctcatcttctgtcattcaaagaagaagtgcgacgaactcgccgcaaagctggtcgcattgggcatcaatgccgtggcctactaccgcggtcttgacgtgtccgtcatcccgaccagcggcgatgttgtcgtcgtggcaaccgatgccctcatgaccggctataccggcgacttcgactcggtgatagactgcaatacgtgtgtcacccagacagtcgatttcagccttgaccctaccttcaccattgagacaatcacgctcccccaagatgctgtctcccgcactcaacgtcggggcaggactggcagggggaagccaggcatctacagatttgtggcaccgggggagcgccctcccggcatgttcgactcgtccgtcctctgtgagtgctatgacgcaggctgtgcttggtatgagctcacgcccgccgagactacagttaggctacgagcgtacatgaacaccccggggcttcccgtgtgccaggaccatcttgaattttgggagggcgtctttacaggcctcactcatatagatgcccactttctatcccagacaaagcagagtggggagaaccttccttacctggtagcgtaccaagccaccgtgtgcgctagggctcaagcccctcccccatcgtgggaccagatgtggaagtgtttgattcgcctcaagcccaccctccatgggccaacacccctgctatacagactgggcgctgttcaaaatgagatcaccctcacacatcccataaccaaattcgtcatggcatgcatgtcggccgacctggaggtcgtcactagcacctgggtgctggtaggcggagtccttgcagctctggccgcatattgcctgacaaccggtagtgtggtcattgtgggtaggatcattttgtccgggaggccggctgttgttcccgacagggaagtcctctaccgggagttcgatgaaatggaagagtgctaactcgag

SEQ ID NO: 6 - DNA, 612 bp, Hepatitus C. Virus
ggatccgatatacatatgggtagtgtggtcattgtgggtaggatcattttgtccggtagtggtagtatcacggcgtacgcccagcagacaaggggcctcctagggtgcataatcaccagcctaactggccgggacaaaaaccaagtggagggtgaggtccagattgtgtcaactgctgcccaaaccttcctggcaacgtgcatcaatggggtgtgctggactgtctaccacggggccggaacgaggaccatcgcgtcacccaagggtcctgtcatccagatgtataccaatgtagaccaagaccttgtgggctggcccgcttcgcaaggtacccgctcattgacaccctgcacttgcggctcctcggacctttacctggtcacgaggcacgccgatgtcattcccgtgcgccggcggggtgatagcaggggcagcctgctgtcgccccggcccatttcctacttgaaaggctcctcggggggtccgctgttgtgccccgcggggcacgccgtgggcatatttagggccgcggtgtgcacccgtggagtggctaaggcggtggactttatccctgtggagaacctagagacaaccatgaggtcctgagaattc

SEQ ID NO: 7 - protein, 4 aa, Artificial Sequence
  Linker sequence between NS3 and NS4A polypeptides
GSGS

SEQ ID NO: 8 - protein, 3 aa, Artificial Sequence
  Peptide sequence attached to prodrug compound activated by hepatitis C virus NS3 protease
SGG

SEQ ID NO: 9 - protein, 3 aa, Artificial Sequence
  Peptide sequence attached to prodrug compound activated by hepatitis C virus NS3 protease
AGG

SEQ ID NO: 10 - protein, 9 aa, Artificial Sequence
  Peptide sequence attached to prodrug compound activated by hepatitis C virus NS3 protease
DEVVPCSGG

SEQ ID NO: 11 - protein, 9 aa, Artificial Sequence
  Peptide sequence attached to prodrug compound activated by hepatitis C virus NS3 protease
DEAVLCSGG

SEQ ID NO: 12 - protein, 9 aa, Artificial Sequence
  Peptide sequence attached to prodrug compound activated by hepatitis C virus NS3 protease
DEVTPCSGG

SEQ ID NO: 13 - protein, 9 aa, Artificial Sequence
  Peptide sequence attached to prodrug compound activated by hepatitis C virus NS3 protease
DEIIVCAGG

SEQ ID NO: 14 - protein, 9 aa, Artificial Sequence
  Peptide sequence attached to prodrug compound activated by hepatitis C virus NS3 protease
DEPLACSGG

SEQ ID NO: 15 - protein, 7 aa, Artificial Sequence
  NON_CONS (4)...(5): Peptide sequence attached to prodrug compound activated by hepatitis C virus NS3 protease
DEFESGG

SEQ ID NO: 16 - protein, 8 aa, Artificial Sequence
  NON_CONS (5)...(6): Peptide sequence attached to prodrug compound activated by hepatitis C virus NS3 protease
DEAVLSGG

SEQ ID NO: 17 - protein, 8 aa, Artificial Sequence
  ACETYLATION (1)...(0)
  NON_CONS (5)...(6): peptide sequence attached to prodrug compound activated by hepatitis C virus NS3 protease
DEVTPSGG

SEQ ID NO: 18 - protein, 8 aa, Artificial Sequence
  Peptide sequence attached to prodrug compound activated by hepatitis C virus NS3 protease
DEIIVAGG

SEQ ID NO: 19 - protein, 8 aa, Artificial Sequence
  Peptide sequence attached to prodrug compound activated by hepatitis C virus NS3 protease
DEPLASGG

SEQ ID NO: 20 - protein, 9 aa, Artificial Sequence
  Peptide sequence attached to prodrug compound activated by hepatitis C virus NS3 protease
DEVVPGSGG

SEQ ID NO: 21 - protein, 7 aa, Artificial Sequence
  Peptide sequence attached to prodrug compound activated by hepatitis C virus NS3 protease
XXVVXCX

(19) United States
(12) Patent Application Publication
(10) Pub. No.: US 2003/0114385 A1
(43) Pub. Date: Jun. 19, 2003

(54) Viral enzyme activated prototoxophores and use of same to treat viral infections

(76) Inventors: Brian E. Cathers, San Diego, CA (US); Saskia T.C. Neuteboon, San Diego, CA (US); H. Michael Shepard, Encinitas, CA (US)

Correspondence Address: Antoinette F. Konski, McCutchen Doyle Brown & Enersen LLP, Suite 1800, Three Embarcadero Center, San Francisco, CA, 94111-4067, US

(21) Appl. No.: 10/133,133

(22) Filed: Apr. 26, 2002

Related U.S. Application Data
(60) Provisional application No. 60/286,893, filed on Apr. 27, 2001.

(51) Int. Cl. A61K 38/08; C07K 7/06; C07K 7/08

(52) U.S. Cl. 514/16; 530/329; 530/328

(57) ABSTRACT

A synthetic prototoxophore, which is a relatively non-toxic compound that includes a toxin moiety and a substrate domain for a viral enzyme, is provided. Upon binding of a viral enzyme to the substrate domain, the catalytic activity of the viral enzyme converts the prototoxophore to a toxophore, which is toxic to a cell. Thus, a toxophore also is provided, as is a pharmaceutical composition containing a prototoxophore, and kits containing a prototoxophore. Also provided are methods of using a prototoxophore to reduce or inhibit viral infectivity, and methods of using a prototoxophore to ameliorate the severity of a viral infection in an individual.

### CROSS-REFERENCE TO RELATED APPLICATIONS

This application claims priority under 35 U.S.C. § 119(e) to U.S. Provisional Application No. 60/286,893, filed Apr. 27, 2001, the content of which is incorporated herein by reference.

### FIELD OF THE INVENTION

The invention relates generally to synthetic compounds that are substrates for viral enzymes, and more specifically to prototoxophores, which are converted upon contact with a viral enzyme to toxophores, to methods of using prototoxophores to prevent or treat a viral infection, and to a means for discovering and testing of hepatitis C virus treatments.

### BACKGROUND

Viral infections are responsible for a variety of diseases as diverse as the common cold, hepatitis, genital herpes, and AIDS. In addition, viruses are opportunistic pathogens that infect individuals in susceptible states due to another disease such as cancer or an autoimmune disease. Viral infections are associated with a great deal of morbidity and mortality throughout the world.

Vaccines are effective against some viral infections, such that an immune response generated by a vaccinated individual prevents or minimizes the severity of an infection by the immunizing virus. In cases such as smallpox, vaccination has essentially resulted in the disappearance of the disease. However, many viruses such as that causing AIDS have been intractable to the development of effective vaccines. In addition, other viruses such as influenza virus change so rapidly that, even when a vaccine is developed that is effective against one strain of the virus, newly developing variant strains render the vaccine useless and, therefore, new vaccines continually must be prepared.

More recently, anti-viral drugs have been developed. One general class of viral drugs are the nucleoside analogs, which interfere with replication of an infecting virus and can be useful against a broad range of viruses. In general, these drugs are not specific for viral replication; they also can affect replication of normal cellular DNA creating adverse side effects. Another class of drugs used against viral infections are the cytokines, including interferons, which can stimulate natural immunity against an infecting virus. Again, however, the effects of cytokines are systemic and, therefore, can produce severe side effects that often result in a patient terminating therapy. Further complicating treatment of viral diseases is the rapid emergence of viruses that are resistance to anti-viral agents (refs. 13 to 31).

Hepatitis C is an example of a viral disease having a clearly unmet need for an effective drug treatment. There are approximately 170 million carriers of the hepatitis C virus (HCV) worldwide (more than four times the number of HIV infected individuals; see refs. 32 and 33). In the United States, alone, there are about 4 million HCV carriers, and mortality due to HCV related liver disease and cancer in the United States is predicted to soon overtake the number of AIDS related deaths (ref. 34).

The hepatitis C virus was discovered in 1988, and in 1989 a diagnostic test for HCV in blood was described (refs. 35 and 36). Testing of the blood supply and of blood donors has dramatically reduced the number of new HCV infections in the United States since 1990, and routine blood testing will increase the number of early diagnoses. However, HCV infection has a long asymptomatic carrier state that can last 10 to 20 years and, therefore, many individuals unknowingly infected prior to 1990 are only now becoming symptomatic.

The acute phase of HCV infection is mild, and about 20% of infected individuals clear the virus from their bloodstream. In the 80% of individuals with persistent infections, chronic active hepatitis ensues and leads to cirrhosis in about 20% of chronically infected people and to hepatocellular carcinoma in another 1% to 5% (ref. 32). In the current approach to HCV diagnosis and treatment, approximately half of those infected will develop chronic active hepatitis and other HCV related complications.

The currently approved therapy for HCV includes a combination of interferon and the antiviral agent ribavirin (ref. 37). Although the clinical response with this new combination is better than interferon alone, these drugs are expensive and treatment is extended over 48 weeks, making patient compliance difficult. Furthermore, as discussed above, these drugs are not selective for virally infected cells, but act generally and systemically. As a result, undesirable side effects occur, including severe flu-like symptoms that cause about 20% of patients discontinuing the therapy. Since these drugs also are used to treat other viral diseases such as AIDS, treatment of the diseases is severely limited due to the consequential systemic toxicity. Thus, a need exists for therapeutic agents that can be used to effectively treat virally infected cells, while sparing uninfected normal cells. The present invention satisfies this need and provides additional advantages.

### DISCLOSURE OF THE INVENTION

The present invention relates to a synthetic prototoxophore, which includes a toxin moiety operatively incorporated into a substrate domain for a viral enzyme, wherein the substrate domain can be bound and modified by the viral enzyme, thereby converting the prototoxophore to a toxophore, which is toxic to a virally infected cell or which reduces or inhibits the production of infectious virions. The substrate domain can be a single domain that is bound by the viral enzyme and modified by a catalytic activity of the viral enzyme, or can be composed of at least a first domain that is bound by the viral enzyme and a second domain that is modified by the catalytic activity of the viral enzyme.

The viral enzyme can be any enzyme that is specifically expressed by a virus, and not generally by a host cell for the virus. For example, the viral enzyme can be a viral protease, which can bind to a first domain of the substrate domain and cleave a second domain, thereby converting a prototoxophore to a toxophore. As such, the substrate domain can be constructed modularly, for example, by including a peptide containing the viral protease cleavage site, and a peptidomimetic that mimics a substrate binding site for the viral enzyme.

The toxin moiety can be any toxin, including, for example, a cytotoxin that can kill or decrease the viability of a host cell for a virus, or a toxin that inhibits a viral process required for maturation of the virus or production of infectious virions. As such, the toxin can be an antimetabolite such as methotrexate, hydroxyurea, 5-fluorouracil, or cytosine arabinoside; an alkylating agent such as nitrogen mustard, cyclophosphamide, melphalan, chloramucin, busulfan or thiotepa; a plant alkaloid such as vincristine, vinblastine, paclitaxel, etoposide, or teniposide; or an antitumor antibiotic such as doxorubicin, daunomycin, idarubicin, bleomycin, mitomycin C, or dactinomycin. Where the toxin is directed to a virally encoded target, it can affect a viral replicase or modify a viral structural protein, thereby reducing or inhibiting viral assembly or the production of infectious virions.

In one embodiment, the toxin moiety of a prototoxophore is a DNA intercalating agent such as mitoxantrone, amsacrine, or a derivative of mitoxantrone or amsacrine, and the prototoxophore contains a substrate domain for a viral protease. For example, the viral protease can be a hepatitis virus protease, a human immunodeficiency virus protease, a rhinovirus protease, a herpes virus protease, an adenovirus protease, or a cytomegalovirus protease. In another embodiment, the viral protease is a hepatitis C virus (HCV) NS3 protease. In another aspect, the prototoxophore has the structure:

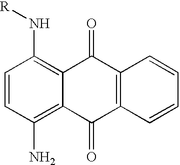

wherein R is (Glu/Asp)-Xaa-Val-Val-(Leu/Pro)-Cys-(Ser/Ala) and wherein Xaa is any any amino acid (Seq. ID No.21), and wherein said compound may be in any enantieric, diasteriomeric, or stereoisomeric form, consisting of a D-form, L-form, α-anomeric form, and β-anomeric form or a pharmaceutically acceptable salt thereof Additional HCV NS3 protease prototoxophores are exemplified by compounds 5761 to 5763 as set forth in Table I, and by compounds 5764 to 5766 and 5768 to 5771 as set forth in Table I.

TABLE I
Peptidyl HCV NS3 protease ECTA substrates.

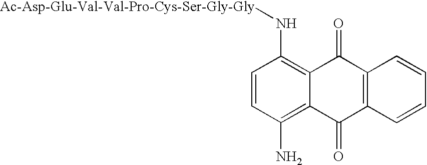

wherein said compound may be in any enantiomeric, diasteriomeric, or
stereoisomeric form, consisting of a D-form, L-form, α-anomeric form,
and β-anomeric form or a pharmaceutically acceptable salt thereof.

| Com- | | Seq. |
|---|---|---|
| pound | | ID |
| # | Peptide sequence | No. |
| 5759 | Ser-Gly-Gly-toxin | 8 |
| 5760 | Ala-Gly-Gly-toxin | 9 |
| 5761 | Acetyl-Asp-Glu-Val-Val-Pro-Cys-Ser-Gly-Gly-toxin | 10 |
| 5762 | Acetyl-Asp-Glu-Ala-Val-Leu-Cys-Ser-Gly-Gly-toxin | 11 |
| 5763 | Acetyl-Asp-Glu-Val-Thr-Pro-Cys-Ser-Gly-Gly-toxin | 12 |
| 5764 | Acetyl-Asp-Glu-Ile-Ile-Val-Cys-Ala-Gly-Gly-toxin | 13 |
| 5765 | Acetyl-Asp-Glu-Pro-Leu-Ala-Cys-Ser-Gly-Gly-toxin | 14 |
| 5766 | Acetyl-Asp-Glu-Phe-Glu-Cha-ABA-Ser-Gly-Gly-toxin | 15 |
| 5767 | Acetyl-Asp-Glu-Ala-Val-Leu-ABA-Ser-Gly-Gly-toxin | 16 |
| 5768 | Acetyl-Asp-Glu-Val-Tbr-Pro-ABA-Ser-Gly-Gly-toxin | 17 |
| 5769 | Acetyl-Asp-Glu-Ile-Ile-Val-ABA-Ala-Gly-Gly-toxin | 18 |
| 5770 | Acetyl-Asp-Glu-Pro-Leu-Ala-ABA-Ser-Gly-Gly-toxin | 19 |
| 5771 | Acetyl-Asp-Glu-Val-Val-Pro-Gly-Ser-Gly-Gly-toxin | 20 |

The structure of the toxin (1,4-diaminoanthraquinone) in a representative ECTA substrate is shown above. Compounds 5759 and 5760 represent the product “toxins” released upon HCV NS3 protease cleavage of the corresponding HCV ECTA substrate (compounds 5761 to 5771). Cha=cyclohexylalanine (3-cyclohexyl-L-alanine), ABA=L-alpha-aminobutyric acid

A synthetic prototoxophore also can contain one or more additional domains of interest, for example, a targeting domain, which can facilitate contact of a prototoxophore with a virally infected target cell. A targeting domain can be any molecule that increases the likelihood that a prototoxophore will contact a target cell, enter the target cell, or localize to a particular compartment within the target cell. Thus, a targeting domain can be a ligand for a cell surface receptor, which is expressed by the target cell, for example, an asialoglycoprotein receptor, which is expressed by hepatocytes, or an epidermal growth factor receptor, which is expressed by epithelial cells, or the like. A targeting domain also can be, for example, an HIV TAT protein transduction domain, which facilitates traversal of a molecule across a cell membrane and into a cell, or can be a cell compartment localization domain. Since a prototoxophore of the invention is not substantially toxic in a cell that is not virally infected, the prototoxophore can comprise any adjuvant that increases access of the prototoxophore to any cell, including, for example, a non-specific cell targeting domain such as a domain comprising a folate derivative, which can target folate receptors expressed by most cell types.

The present invention also relates to compositions comprising a synthetic prototoxophore, including, for example, a prototoxophore contained in a liposome, a pharmaceutical composition containing a prototoxophore, and the like. Kits containing a prototoxophore or composition containing a prototoxophore also are provided.

The present invention also relates to a toxophore, which is produced by contacting a synthetic prototoxophore of the invention with a viral enzyme having catalytic activity of the prototoxophore substrate domain, under conditions that allow the viral enzyme to modify the prototoxophore. In one embodiment, the toxophore is produced due to modification of a prototoxophore by a viral protease. Such a toxophore is exemplified by a toxophore produced due to cleavage of a prototoxophore by the HCV NS3 protease, for example, a toxophore having the structure of the product shown in FIG. 4, or as compound 5759 or 5760 as shown in Table I.

The present invention further relates to a method of reducing or inhibiting viral infectivity. Such a method can be performed, for example, by contacting a cell, which is infected with a virus or is susceptible to infection with a virus, with an amount of a synthetic prototoxophore that, when converted by a viral enzyme to a toxophore, is sufficient to decrease the viability of a cell containing the viral enzyme or to decrease the ability of the virus to produce infectious virions. The cells can be cell lines, for example, which have been adapted to long term continuous culture, in which case the cells are contacted with the prototoxophore in cell culture. The cells also can be cells of an individual, which can be removed or isolated from the individual and contacted with the prototoxophore ex vivo, or can be contacted in situ in the individual or in a portion of the individual isolated, for example, by a shunt. The virus can be any virus that expresses an enzyme that can convert a prototoxophore to a toxophore, particularly a viral enzyme that converts the prototoxophore to a toxophore to a substantially greater extent than may a cellular enzyme.

The cell to be contacted with the prototoxophore, and which can be infected with a virus, can be a cell of any eukaryotic organism, particularly a mammalian cell such as a human lymphocyte, nerve cell, connective tissue cell, muscle cell, or epithelial cell. In one embodiment, the cell to be contacted is a hepatocyte. In another embodiment, the viral enzyme, which converts the prototoxophore to a toxophore, is a viral protease, for example, a hepatitis C virus NS3 protease, and the prototoxophore is any of compounds 5761 to 5763 as set forth in Table I or any of compounds 5764 to 5766 and 5768 to 5771 as set forth in Table I.

The present invention also relates to a method of ameliorating the severity of a viral infection in an individual. Such a method can be performed, for example, by administering to the individual an amount of a synthetic prototoxophore that, when converted by a viral enzyme to a toxophore in a cell in the individual, is sufficient to decrease the viability of the virally infected cell or to reduce or inhibit the ability of the virus to produce infectious virions. The prototoxophore can include a targeting domain, for example, a ligand for a cell surface receptor expressed by cells infected or susceptible to infection by the virus, or the prototoxophore can be contained in a liposome, which can be formulated to contain a ligand for the cell surface receptor.

The viral infection can be any viral infection, including a mammalian viral infection, and particularly a human viral infection. The virus can be any virus, particularly a virus that causes or exacerbates a disease, for example, a human immunodeficiency virus (HIV) such as HIV-1, a herpes simplex virus (HSV) such as HSV-1 or HSV-2, a rhinovirus infection, or a hepatitis virus. In one embodiment, the virus is a hepatitis C virus (HCV), the viral enzyme is the HCV NS3 protease, and the prototoxophore has a structure of any of compounds 5761 to 5763 as set forth in Table I or any of compounds 5764 to 5766 and 5768 to 5771 as set forth in Table I. In another embodiment, the prototoxophore also contains a targeting domain comprising a ligand for an asialoglycoprotein receptor.

A method of the invention can be performed by administering the prototoxophore as a single treatment, either as a bolus injection or by infusion over a period of time, or can be performed as a series of separate treatments over a period of time, including days, weeks, or longer. In addition, a method of the invention is suitable for use as one modality of a combined modality treatment. As such, the individual also can be treated with one or more therapeutic agents that are directed to alleviating the signs or symptoms of a pathologic condition associated with the viral infection. For example, in addition to administration of a prototoxophore, the individual can be treated with an antibiotic or antifungal agent to treat or prevent opportunistic infections by bacteria, fungi, protozoans, or other microbial organisms where the individual has or is susceptible to an opportunistic infection, or can be treated with a cancer chemotherapeutic agent where the individual is suffering from a cancer.

Further provided is an assay to identify anti-viral agents, comprising contacting a virally infected cell with an amount of a candidate agent and comparing the ability of the candidate agent to inhibit the growth or infectivity of the virus in said virally infected cell to the ability of a compound of claim 1 to inhibit the growth or infectivity of the virus in said virally infected cell. Suitable virus and virally-infected cells are as described herein for a compound of this invention. Candidate agents with the same or similar therapeutic activity are useful in the methods disclosed for a compound of this invention.

### BRIEF DESCRIPTION OF THE DRAWINGS

FIG. 1 illustrates the fragments produced upon HCV polyprotein processing. Structural polypeptides include capsid (C), envelope proteins (E1 and E2), and a protein of unknown function (p7). Non-structural (NS) polypeptides include the Metallo/Cysteine protease (2); the mixed function serine protease and NTPase/Helicase (3); the NS3 protease cofactor (4A); a protein of unknown function (4B); a protein that confers interferon resistance (5A); and the RNA-dependent RNA polymerase (5B).

FIG. 2 shows a representation of an HCV NS3 protease ECTA compound (prototoxophore).

FIG. 3 shows the parent drug molecules, Mitoxantrone and Amsacrine, and analogues and derivatives thereof, some of which contain a peptide or peptide-like motif.

FIG. 4 shows a peptidic prototoxophore and an HCV NS3 protease cleavage product (toxophore). The released toxophore is an analogue of the anti-cancer agent, mitoxantrone.

FIG. 5 shows a peptidomimetic HCV ECTA molecule. The toxin moiety is related to mitoxantrone, and the linker is no longer a tripeptide (compare FIG. 4). In the exemplified prototoxophore, the mitoxantrone derivative contains an amine group substituted for the hydroxyl group at the site where the peptidomimetic is bound to the toxin moiety, thereby operatively incorporating the toxin moiety and substrate domain.

FIG. 6 provides an example of a prototoxophore comprising a D-galactopyranose-HCV ECTA conjugate. The linker can be a peptide or a peptidomimetic.

FIGS. 7A to 7C show the results of DNA titration studies using HCV ECTA compounds. FIG. 7A—10 μM HCV ECTA compound 5759 following 15 min. incubation with DNA. FIG. 7B—12 μM compound 5763 following 15 min incubation with DNA. FIG. 7C—12 μM compound 5764 following 5 hr incubation with DNA. HCV ECTA compounds are shown in Table I. DNA units refer to μM of intercalator binding sites.

FIG. 8 illustrates a fusion protein designed to detect HCV NS3 protease activity in cells. “GFP” indicates green fluorescent protein; “FLAG” indicates the FLAG epitope. The NS3 cleavage site is indicated.

### MODES FOR CARRYING OUT THE INVENTION

The present invention provides synthetic prototoxophores that are substrates for viral enzymes and are converted by specific viral enzymes to active toxophores, which are toxic to an infected cell containing the viral enzyme or which reduce or inhibit the ability of the infecting virus to produced infectious virions. As disclosed herein, an Enzyme Catalyzed Therapeutic Activation (ECTA) approach was used to identify viral enzyme activated prototoxophores, which can be used as therapeutic agents for treating or preventing a viral infection. Accordingly, the present invention also provides methods for reducing or inhibiting viral infectivity by contacting a cell infected with or susceptible to infection by a virus with a prototoxophore, whereby an enzyme expressed by the virus in the cell converts the prototoxophore into a toxophore, which is toxic to the infected cell in which the viral enzyme is expressed or which reduces or inhibits the production of infectious virions in the infected cell. The present invention also provides a method of amelioriating the severity of a viral infection in a subject having or susceptible to a viral infection by administering a prototoxophore to the subject, whereby, in the presence of an enzyme expressed by a virus in a cell in the subject, the prototoxophore is converted into a toxophore, which is toxic to cell.

The ECTA approach provides a means to treat a variety of diseases including cancers and infectious diseases by allowing the production of compounds (referred to as ECTA compounds, or prototoxophores) that act as substrates for an enzyme that is expressed primarily in cells associated with the disease, but not in normal healthy cells, whereby an enzyme-catalyzed transformation of the prototoxophore results in the generation of a toxic reaction product (toxophore). As such, an ECTA compound is distinguishable, for example, from compounds that act by inhibiting the activity of an enzyme. An ECTA compound provides the advantage that the prototoxophore is relatively non-toxic to uninfected cells. In addition, the toxophore is generated primarily inside a diseased cell due to activation by the target enzyme, thereby directing toxicity to the diseased cell and reducing or eliminating any systemic toxicity that otherwise would be caused by administration of the toxin, itself.

The ECTA approach has been applied to cancer by targeting thymidylate synthase (see ref. 1) and to drug resistant bacterial infections by targeting β-lactamase (see U.S. Pat. No. 6,159,706 and PCT International Application Nos. PCT/US01/06519 and PCT/US01/14133). As disclosed herein, enzymes expressed only in virus infected cells represent a distinct opportunity for ECTA technology because many viral pathogens express viral specific enzymes such as proteases, which are essential to the maturation and formation of new viral particles (see Table II; see, also, refs. 2 and 3). The unique characteristics of the viral proteases have been used to design potent and selective viral enzyme inhibitors, many of which have been approved for drug use (ref. 3). The ECTA approach provides a means to identify a new class of therapeutic agents useful for viral disease by targeting virally encoded enzymes such as proteases, which can catalytically modify a prodrug (prototoxophore) to an active therapeutic agent (toxophore). As disclosed herein, the activated toxophore can be toxic to the virally infected cells or can block production of infectious virions.

TABLE II
Viral pathogens and virally encoded proteases necessary for
viral maturation.

| | Condidate ECTA |
|---|---|
| Virus | Proteases |
| Hepatitis C Virus | NS3 Protease |
| Human Immunodeficiency Virus | HIV Protease |
| Cytomegalovirus | CMV Protease |
| Herpes Simplex Virus | HSV Protease |
| Adenovirus | L3 Protease |
| Rhinovirus | 3C and 2A proteinase |

Protease activated prodrugs have been examined as anti-cancer agents (see refs. 87 to 97). However, the viral enzyme ECTA approach disclosed herein provides several advantages over methods that would simply adapt the anti-cancer methods to treating a viral infection. For example, the ECTA compounds (prototoxophores) target viral specific enzymes such as viral proteases rather than more ubiquitously expressed proteases such as carboxypeptidase A (see ref. 90) or plasmin (see refs. 91 and 92). As such, prototoxophores can provide a better therapeutic index as compared to those obtained using cancer prodrug approaches. In addition, ECTA compounds target intracellular proteases, not extracellular proteases such as prostate specific antigen (PSA; refs. 87 to 89), plasmin or carboxypeptidase A and, therefore, should be associated with a diminished bystander effect (see ref. 90), which, while useful for treating a cancer, would result in harm to otherwise healthy non-virally infected cells where the goal is treatment of a viral infection. The toxin moiety of a prototoxophore also need not simply be a cytotoxic compound, as with cancer chemotherapeutic drugs, but also can be a toxophore that inhibits a critical viral process and reduces or inhibits maturation of the virus or production of infectious virions. An additional advantage is that the substrate domain of a prototoxophore does not have to be attached to the toxin moiety via a self-immolative (traceless) linker. As such, a broader range of linkers are available for use in constructing a prototoxophore of the invention as compared, for example, to the self-immolative linkers used to prepare cancer chemotherapeutic agents such as a prostate-specific antigen binding element and cleavage site linked through a self-immolative linker to daunomycin. Also, the ECTA approach does not require the antibody-mediated delivery of an activating enzyme to the target cancer as with the antibody-directed enzyme prodrug therapy (ADEPT) approach to cancer therapy and does not require the introduction of a polynucleotide encoding a bacterial enzyme using a gene therapy procedure as required of the virus-directed enzyme prodrug therapy (VDEPT) approach to cancer therapy (refs. 94 to 97).

Accordingly, the present invention provides synthetic prototoxophores, which include a toxin moiety operatively incorporated into a substrate domain for a viral enzyme. As used herein, the term “prototoxophore” refers to a compound that is relatively non-toxic to cells, particularly cells of a vertebrate, including a mammal, and that can be converted due to the catalytic activity of a viral enzyme to a toxophore, which is relatively toxic to cells or which reduces or inhibits the production of infectious virions in the infected cell. The term “toxophore” is used herein to refer to the product of viral enzyme activity on a prototoxophore. It will be recognized that the toxicity of a prototoxophore and a toxophore are compared to each other, thereby allowing a determination that a prototoxophore is relatively non-toxic with respect to a toxophore generated therefrom. In addition, the toxicity of a prototoxophore can be compared to that of a free form of the toxin moiety of the prototoxophore, and determining that the prototoxophore is relatively less toxic to cells than the free form of the toxin moiety (see Example 1).

A prototoxophore generally has an IC₅₀ (concentration that inhibits the growth of 50% of cells) or an LD₅₀ (dose that kills 50% of cells) that is at least about two-fold greater than the IC₅₀ or LD₅₀ of a toxophore generated therefrom, usually at least about three-fold greater, particularly at least about five-fold greater, and preferably at least about ten-fold greater than the IC₅₀ or LD₅₀ of a toxophore generated therefrom. Methods for determining the IC₅₀ or LD₅₀ or other such indicator of toxicity of cells in culture are disclosed herein (Example 1C) or otherwise known in the art. Similarly, toxicity can be determined in experimental animals, for example, an LD₁₀, LD₅₀, or the like, using routine methods.

A synthetic prototoxophore of the invention includes a toxin moiety operatively incorporated into a substrate domain for a viral enzyme. As used herein, the term “operatively incorporated into” means that the components of a prototoxophore, including the toxin moiety, the substrate domain, and any other domain, are associated in a form that is stable under physiological conditions, that is accessible to modification by a target viral enzyme, and that results in the formation of a toxophore upon modification by the viral enzyme. As such, the toxin moiety can be one that is released following rearrangement upon viral enzyme modification such as occurs in NB2001 (see PCT International Application No. PCT/US01/14133), or can be inherent such as the toxin moiety of NB 1011 (see ref. 1). In one embodiment, the toxin moiety and substrate domain are covalently linked through a chemical bond. However, the toxin moiety and substrate domain, or any other domain or molecule that is a component of the prototoxophore also can be associated, for example, through a noncovalent interaction such as a biotin/avidin linkage or any other such interaction that is stable under physiological conditions and that does not interfere with the ability of the target viral enzyme to effect its catalytic activity.

As used herein, the term “target viral enzyme” refers to a viral enzyme that can specifically bind the substrate domain of a particular prototoxophore and that can effect its catalytic activity on the prototoxophore. In general, a target viral enzyme is expressed only in a virally infected cell and, as such, conversion of a prototoxophore to an activated toxophore only occurs in the target cell, which is then killed by the toxophore. The term “target cell” is used herein to refer to a cell that is infected by a virus, or that is susceptible to infection by a virus, and in which the prototoxophore is converted to a toxophore due to catalytic activity of the target viral enzyme.

A target viral enzyme can be any enzyme that is encoded by a virus and is expressed in cells that have been infected by the virus, but not generally in cells that are not infected by the particular virus. Thus, the viral enzyme can be a viral protease as exemplified in Table II or otherwise known in the art, including an RNA-dependent RNA polymerase, a reverse transcriptase, an integrase, or the like.

A synthetic prototoxophore, which can be converted to a toxophore by the catalytic activity of a viral enzyme, contains at least two operatively incorporated components—a toxin moiety and a substrate domain (see FIG. 2). As used herein, the term “substrate domain” refers to a region of a prototoxophore that is bound by an enzyme and is modified due to the catalytic activity of the enzyme. Since the substrate for viral enzymes generally are proteins, a substrate domain is conveniently discussed with reference to a polypeptide sequence. As disclosed herein, however, a substrate domain can comprise, in whole or in part, a polypeptide, peptidomimetic, polynucleotide, small organic molecule, or the like.

It is recognized that the portion of a substrate that is bound by an enzyme can, but need not be, the same sequence or a sequence contiguous with the portion that is modified by the enzyme. As such, a substrate domain of a prototoxophore can comprise a single domain, which is both bound by and modified by the viral enzyme, or can comprise two or more domains such as a recognition domain, which is bound by the enzyme, and a modification domain, which is modified due to the catalytic activity of the viral enzyme. For example, where the viral enzyme is a viral protease, it can specifically bind to a first domain of the substrate domain and cleave a second domain. As such, a substrate domain can be considered a modular structure, which can comprise, for example, with reference to a viral protease, a peptide domain containing the viral protease cleavage site, and a peptidomimetic domain that mimics a substrate binding site for the viral enzyme.

As used herein, reference to a substrate domain being “bound” or “specifically bound” by a viral enzyme means that the enzyme interacts with the substrate domain with sufficient specificity such that the enzyme can effect its catalytic activity on a prototoxophore comprising the substrate domain under physiological conditions, or conditions that substantially mimic physiological conditions. Conditions that substantially mimic physiological conditions can be cell culture conditions or in vitro conditions that account for buffer capacity, pH, salt concentration, ionic strength, and the like, and that allow for a target viral enzyme to effect its catalytic activity on a naturally occurring substrate for the enzyme. In general, specific binding of a target viral enzyme and a prototoxophore is characterized, in part, by a catalytic efficiency (kcat/Km) of at least about 100 M⁻¹s⁻¹, generally at least about 1000 M⁻¹s⁻¹, and usually at least about 10,000 M⁻¹s⁻¹. In addition, a prototoxophore of the invention is characterized in that it is preferentially converted to a toxophore due to the action of a viral enzyme as compared to a cellular enzyme that may have a similar substrate specificity as the viral enzyme specificity. Thus, prototoxophore of the invention in characterized, in part, in that a viral enzyme converts the prototoxophore to a toxophore with at least about a two-fold greater efficiency, generally at least about a five-fold greater efficiency, and particularly at least about a ten-fold greater efficiency than does a cellular enzyme.

As used herein, reference to a substrate domain being “modified” or “converted” by a viral enzyme means that the viral enzyme has effected its catalytic activity on the substrate domain. Thus, depending on the catalytic activity of the viral enzyme, a modified substrate domain can be a domain that has been phosphorylated (a viral enzyme kinase), glycosylated (a viral enzyme glycosyltransferase), cleaved (a viral enzyme protease or a viral enzyme endonuclease), or the like. Methods for identifying such modifications are routine and well known in the art (see, also, Examples 1B and 1D).

A substrate domain of a prototoxophore can be a naturally occurring substrate for a particular viral enzyme, or a peptide portion of the naturally occurring substrate that can be bound and modified by the enzyme, or can be a peptide or other molecule that is derived from or based on the naturally occurring substrate. For example, a substrate domain of a hepatitis C virus (HCV) NS3 protease is exemplified herein by peptides based on a consensus sequence determined to be more active than the natural cleavage sites in the HCV polyprotein and on sequences determined to be resistant to proteolysis by yeast cell proteases (see Table I; see, also, ref. 79). As disclosed herein, the substrate domain also can be a synthetic molecule that has a structure that mimics the structure of a substrate for the viral enzyme, for example, a peptidomimetic (refs. 4 to 15) or a polynucleotide (see below).

The toxin moiety of a prototoxophore of the invention can be any toxin, particularly a cytotoxin that can kill or decrease the viability of a target cell, including any agent generally used as a therapeutic agent. Thus, the toxin moiety can be, for example, an agent that disrupts the structure or function of DNA in a cell, including cellular DNA, viral DNA or both; an agent that disrupts a metabolic pathway in a cell; an agent that induces cellular apoptosis or necrosis; an agent that disrupts that ability of a cell to traverse the cell cycle, including agents that inhibit DNA synthesis or mitosis; or any other specific or pleiotropic toxic agent (see below). The toxophore also can target a viral component. For example, the production of pyridinioalkanoyl thioesters are known to inactivate HIV-1 nucleocapsid protein P7 (ref. 109). Phosphonoformate and derivatives are effective inactivators of viral-encoded DNA polymerase and reverse transcriptase (ref. 110). Toxophores that prevent the function of a viral RNA-dependent RNA polymerase such as the HCV RNA-dependent RNA polymerase can be based on nucleotide analogs, including, for example, direct inhibitors such as modified nucleotides or modified phophonoformate, or indirect inhibitors such as flavopiridol (see, for example, ref. 111).

Toxin moieties useful in a prototoxophore of the invention include, for example, antimetabolites such as methotrexate, hydroxyurea, 5-fluorouracil, or cytosine arabinoside; alkylating agents such as nitrogen mustard, cyclophosphamide, melphalan, chloramucin, busulfan or thiotepa; plant alkaloids such as vincristine, vinblastine, paclitaxel, etoposide, or teniposide; and antitumor antibiotics such as doxorubicin, daunomycin, idarubicin, bleomycin, mitomycin C, or dactinomycin (see, for example, Harrison's “Principles of Internal Medicine” 13th ed. (ref. 112). The general classes of cytotoxic molecules useful as toxin moieties, inclusive of the above, include, for example, indolocarbazoles, imidazotetrazines, anthracyclins, mitomycins, bleomycins, cytotoxic nucleosides, pteridine family, nitrogen mustards, diynes, podophillotoxins, and taxoids, and additional specific examples of cytotoxic agents within these classes, include, for example, mitozolomide, carminomycin, daunorubicin, aminopterin, methopterin, dichloromethotrexate, porfiromycin, 6-mercaptopurine, podophillotoxin, etoposide phosphate, vindesine, combretastatin, camptothecin, apoptolidene, epothilone, halichondrin, hemiasterlin, methioprim, thapsigargin, chloroquine, and 4-hydroxycyclophosphamide.

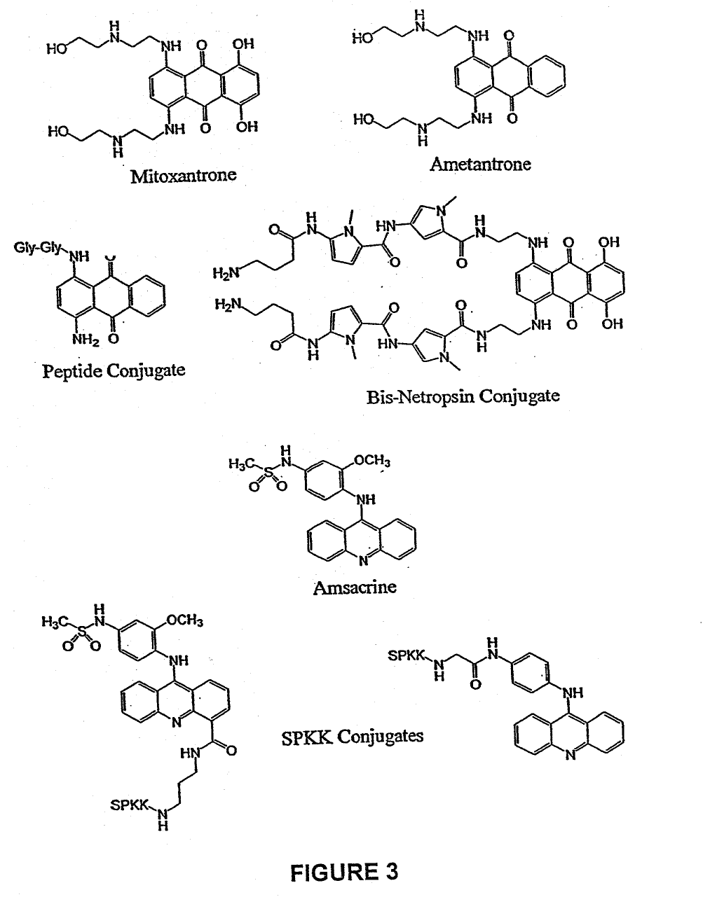

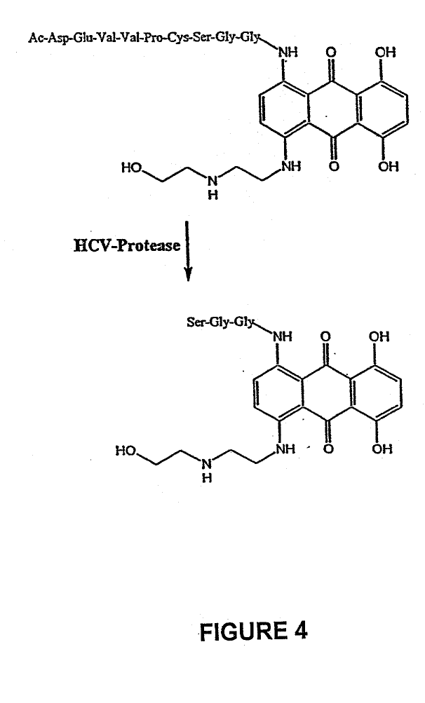

Mitoxantrone and m-AMSA were selected for the studies disclosed herein, in part, because they can be modified with peptide or peptide-like moieties and still retain their cytotoxic properties (see FIG. 3; see, also, refs. 68 to 76). The ability to retain cytotoxic activity even when so modified was considered because the result of catalytic activation of an HCV NS3 protease prototoxophore is the presence of a small peptide, chemical linker, or the like, attached to the toxin moiety following cleavage of the prototoxophore by the protease (see FIG. 4). Thus, where the activation of a prototoxophore by a viral enzyme results in a toxophore comprising a peptide or other chemical group linked to the toxin moiety, the toxin moiety is selected based on its ability to maintain potency when so modified.

As disclosed herein, a prototoxophore acts as a substrate for a viral enzyme such that the prototoxophore, which is relatively non-toxic to a host cell, is converted to a toxophore, which can be toxic to a virally infected cell or can reduce or inhibit production of infectious virions. Depending on the activity of the particular viral enzyme, the toxophore can comprise the entire structure of the prototoxophore, and can further include a modification due to the enzyme activity, for example, the addition of one or more phosphate groups, glycosyl groups, or the like, such that the prototoxophore is converted to a toxophore. A toxophore also can comprise a portion of the prototoxophore, for example, a cleavage product of the prototoxophore comprising the toxin moiety and a peptide or oligonucleotide sequence. A toxophore also can comprise only the toxin moiety, for example, where the catalytic activity of the viral enzyme results in release of the toxin moiety from the prototoxophore. Since the conversion of a prototoxophore to a toxophore depends on the catalytic activity of the viral enzyme, the toxic effect is limited primarily to those cells expressing the viral enzyme, although it is recognized that, due to death of the target cells, some active toxophore may be released at the site of the cell death or into the circulation. However, the amount of such release is expected to be minimal and insufficient to produce significant localized or systemic cell killing.

Methods for determining that a compound such as a toxophore is toxic to a cell, or for confirming that a compound such as a prototoxophore lacks toxicity or has a sufficiently low level of toxicity, are disclosed herein or otherwise known in the art. For example, an agent to be examined for toxicity, for example, a prototoxophore or an activated form of the prototoxophore, simply can be contacted with cells in culture and any change in survival of the cells can be monitored, or toxicity tests in experimental animals can be performed (see Examples 1C and 1E). In addition, a prototoxophore or an activated form thereof can be examined to determine whether the toxin moiety is acting in the manner in which it is known to exert its toxic effect. For example, as disclosed herein, an activated form of a prototoxophore comprising a DNA intercalating agent as a toxin moiety was examined for DNA intercalating activity, which is indicative of the toxin activity and, in fact, correlated with cytotoxicity (see Examples 1A and 1C).

Where a toxophore is expected to act by inducing apoptosis of a target cell, i.e., comprises a toxin moiety known to induce apoptosis, a cell contacted with the toxophore (or prototoxophore) can be monitored for the characteristic structural changes due to apoptosis in a cell, including, for example, disruption of the cytoskeleton, cell shrinkage, membrane blebbing, or nuclear condensation due to degradation of DNA; or by detecting the characteristic nucleosomal degradation pattern of genomic DNA by gel electrophoresis, or the like. In addition, commercially available assays to detect, for example, annexin V binding to a cell in conjunction with propidium iodide exclusion; mitochondrial membrane potential disruption; poly (ADP-ribose) polymerase activity; and the like (R & D Systems, Minneapolis Minn. Alexis Biochemicals, San Diego Calif.) can be used is an indicator of apoptosis. Thus, ECTA compounds having desirable level of toxicity in a prototoxophore form and an activated toxophore form can be identified and differentiated.

A synthetic prototoxophore also can contain one or more additional domains, depending, for example, on the target cell and the target viral enzyme. For example, the prototoxophore can comprise a targeting domain, which can facilitate contact of the prototoxophore with a particular target cell, transport of the prototoxophore into the cell, localization of the prototoxophore in an intracellular compartment containing the target viral enzyme.

Since prototoxophores are not substantially toxic absent conversion by a viral enzyme into an active toxophore, it is not necessary to target prototoxophores to virally infected cells. Nevertheless, targeting prototoxophores to particular cells likely to be infected by a virus can reduce the amount of a prototoxophore that must be administered to an individual, for example, to treat a viral infection and, therefore, can substantially reduce the cost of treatment to the individual. Thus, a prototoxophore can comprise a targeting domain such as a ligand for a cell surface receptor that is expressed by the target cell (or a receptor domain, where the cell expresses a ligand on its surface). Cell surface ligands and receptors are well known in the art and include, for example, an asialoglycoprotein receptor, which is expressed by hepatocytes, an epidermal growth factor receptor, which is expressed by epithelial cells, and the like. Since a prototoxophore of the invention is not substantially cytotoxic absent modification to a toxophore by a viral enzyme, the prototoxophore can comprise any adjuvant that increases the likelihood of contact of the prototoxophore to any cell, including the virally infected cells. As such, a targeting domain can be a non-specific cell targeting domain, in that it targets a broad range of cell types that, for example, express a common receptor. For example, a non-specific cell targeting domain of a prototoxophore can comprise a folate derivative, which can target folate receptors, which are expressed by most cell types.

Mammalian hepatocyte plasma membranes, for example, contain the asialoglycoprotein receptor (ASGP-R; refs. 80 to 82), which is a unique integral membrane bound receptor that is specific for terminal, nonreducing, D-galactopyranosyl or 2-acetamido-2-deoxy-D-galactopyranosyl residues. In humans, binding to the ASGP-R occurs rapidly, within one minute, and endocytosis occurs within two minutes, with recycling of the receptor occurring in seven minutes (ref. 83). It has also been demonstrated in vitro and in vivo that synthetic galactose polymer ligands are taken up to a significant extent by ASGP-R expressing cells (refs. 84 to 86). Thus, where it is desired to target a viral infection of the liver, a prototoxophore can comprise a targeting domain that includes D-galactopyranose residues, thereby increasing the likelihood that the prototoxophore will contact target hepatocytes. An example of such a prototoxophore, which can be activated by the HCV NS3 protease to a toxophore is shown in FIG. 6.

In many cases, the peptide sequences, polynucleotide sequences, organic linkers, peptidomimetic or other molecule comprising a prototoxophore is selected such that the prototoxophore is suitable for use as a therapeutic. However, the chemical nature of the substrate domain or toxin moiety or both, for example, the net charge, or hydrophobicity or hydrophilicity, or simply the molecular weight of the prototoxophore, may impede the ability of the molecule to enter a target cell. Accordingly, a prototoxophore can contain a targeting domain that facilitates traversal of the prototoxophore through a cell membrane and into a cell, including the target cell. Such a targeting domain can be, for example, an HIV TAT protein transduction domain, which facilitates traversal of a molecule across a cell membrane and into a cell (see refs. 113, 114), or can be a cell compartment localization domain. Alternatively, or in addition, the prototoxophore can be formulated such into a composition that facilitates entry into a cell, for example, by incorporation into a liposome (see below).

Where the viral enzyme is expressed in or localizes to a particular cellular compartment, the prototoxophore can include a cell compartmentalization domain such that the prototoxophore is directed to the same intracellular compartment containing the target viral enzyme. Cell compartmentalization domains are well known and include a plasma membrane localization domain, a nuclear localization signal, a mitochondrial membrane localization signal, an endoplasmic reticulum localization signal, and the like (see, for example, refs. 115, 116 and U.S. Pat. No. 5,776,689).

A viral protease provides a useful target for illustrating the ECTA approach for generating prototoxophores that target a viral enzyme because the substrates for viral proteases generally are very large proteins and the active site of the viral proteases can accommodate large moieties positioned proximal to the scissile bond. Accordingly, the present invention is exemplified by using an ECTA approach to identify prototoxophores that target the NS3 protease of hepatitis C virus (HCV; Example 1). Unlike previously described compounds that act as inhibitors of the HCV NS3 protease (see refs. 39, 49, 50), the HCV prototoxophores of the invention take advantage of the NS3 protease activity, which converts the prototoxophore to a toxophore (toxin), thereby selectively killing cells expressing the viral enzyme.

HCV is a member of the Flaviviridae family of viruses that have in common an enveloped nucleocapsid containing a single (+)-stranded RNA genome (ref. 38). The HCV genome is approximately 9.4 kb in length and encodes a polyprotein of about 3000 amino acids (ref. 39). HCV infection is prevalent, and has been refractory to treatment. One approach to treatment has been the use of peptides or synthetic molecules that act to inhibit HCV protease activity, which is necessary for viral infectivity. Another approach to HCV therapy has been directed to the development of a vaccine to prevent the spread of the disease or to invoke an immune response sufficient to rid the host of the infection (therapeutic vaccination; ref 60). However, the development of standard vaccines using coat proteins has been difficult due to the tremendous variability and high mutational frequency of the coat proteins, but some success has been achieved (ref. 61). Yet another approach has been directed to developing a DNA vaccine (refs. 62 to 65). DNA vaccines can elicit a much stronger cellular immune response than can standard vaccines. By activating cytotoxic T lymphocytes (CTLs) toward killing HCV infected cells, the disease could be halted. However, there has been only marginal success in developing a vaccine for HCV (see ref. 60).

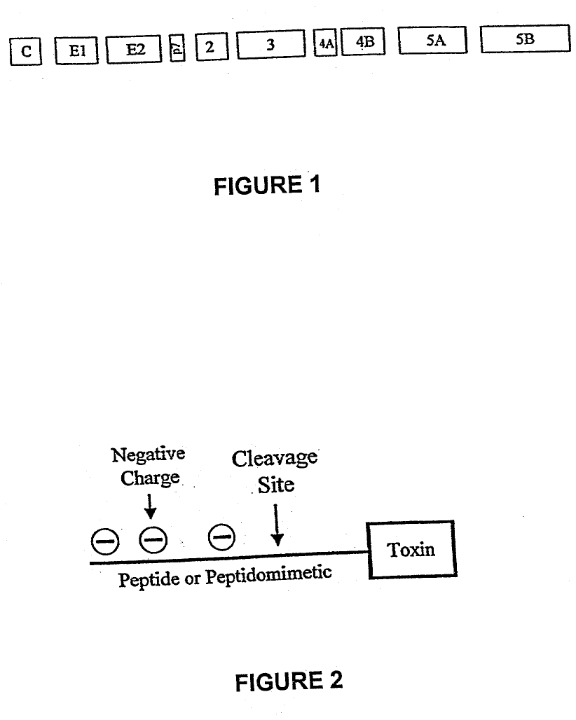

A prototoxophore of the invention can be used to reduce HCV infection by targeting an HCV viral protease such as the HCV NS3 protease in a manner unlike previous approaches. The HCV viral polyprotein is cleaved co-translationally and post-translationally by host and virally encoded proteases. Within the polyprotein, the structural proteins are located at the N-terminal quarter and the non-structural (NS) proteins make up the remainder (see FIG. 1). The cleavage sites in the N-terminus of the HCV polyprotein are believed to be processed by host signal peptidases (refs. 40 to 43). The NS2/3 site is autocatalytically cleaved by a metalloproteinase or a cysteine protease that includes parts of NS2 and NS3 (refs. 44 to 47). The N-terminus of the NS3 protein contains a serine protease that is responsible for processing the viral polyprotein at the NS3/4A, NS4A/4B, NS4B/5A, and NS5A/5B junctions, and the C-terminus acts as a bifunctional NTPase/helicase that may be involved in viral RNA replication (refs. 48, 51 to 54).

The serine protease domain of the NS3 protein contains the “classical” chymotrypsin-like fold and catalytic triad (ref. 55). Unlike classical serine proteases, however, the NS3 has a strict requirement for a peptide cofactor (NS4; ref. 56), and is active as a heterodimer comprising amino acids 1 to 180 of the HCV NS3 gene product and the 54 amino acid NS4A gene product (referred to herein as “NS3 protease”; see refs. 48, 56, 66 and 76). In addition, the HCV NS3 protease requires a structural zinc ion, and garners substrate binding affinity through extended backbone interactions as opposed to side chain interactions (refs. 57 to 59). This last feature has made the design of potent and selective NS3 protease inhibitors especially difficult.

The components of a prototoxophore for targeting HCV NS3 protease include a toxin moiety and substrate domain as illustrated generally in FIG. 2. The substrate domain includes a negatively charged component that allows for specific binding by the protease and contributes to selectivity and potency of the prototoxophore, and a cleavage site for the protease. The binding region and cleavage site of the substrate domain can be derived from a contiguous part or separate parts of a naturally occurring NS3 protease substrate, can be selected based on the structures of known potent and selective inhibitors of HCV NS3 protease (see ref. 49), or can be selected, for example, from a library of peptidomimetics or other such molecules prepared by combinatorial methods or designed based on known properties required of the particular binding domain. It is known, for example, that the negative charge on the non-prime side of the cleavage site is important for avid binding of the substrate by the NS3 protease (ref. 66) and, therefore, a component having this characteristic was included in the substrate domain of the exemplified HCV NS3 protease prototoxophores (see, for example, Table 2 and FIGS. 4 to 6). As disclosed herein, this negatively charged portion provides the additional advantage of contributing to the relatively non-toxic character on an NS3 prototoxophore (see Examples 1A and 1C). The presence of a cleavage site for the NS3 protease distinguishes a prototoxophore of the invention from drugs designed to be inhibitors of enzymes in that chemically stable groups generally are substituted for the scissile bond of the substrate in inhibitory molecules. In contrast, the scissile bond is maintained intact in a prototoxophore of the invention such that, upon contact with an NS3 protease, the prototoxophore is cleaved and a toxophore, which is substantially more toxic than the prototoxophore, is generated.

As disclosed herein, the ECTA approach has been used to identify prototoxophores that target the HCV NS3 protease (see Example 1). Thus, in one embodiment, the toxin moiety of a prototoxophore is a DNA intercalating agent such as mitoxantrone, amsacrine, or a derivative of mitoxantrone or amsacrine, and the substrate domain is a substrate for the HCV NS3 protease. Such prototoxophores are exemplified by compounds 5761 to 5763 as set forth in Table I, and by compounds 5764 to 5766 and 5768 to 5771 as set forth in Table I. Furthermore, in view of the exemplified prototoxophores, it will be recognized that the ECTA approach can be used to successfully identify prototoxophores that are activated to toxophores by other viral enzymes such as those listed in Table I and, therefore, to identify therapeutic agents that selectively exert their effect in virally infected cells.

The criteria for characterizing and selecting prototoxophores that target viral enzymes include selectivity, potency, efficacy and latency of the prototoxophore. Selectivity is characterized by the level of preferential or specific activation of the prototoxophore, i.e., conversion to the toxophore, in a virally infected cell due to the catalytic activity of an enzyme expressed specifically by the virus. Potency is characterized by the avidity and velocity of the interaction of the prototoxophore with the viral enzyme such that the interaction can effectively compete with other cellular processes, including the activity of the viral enzyme for its natural target, for example, the NS3 protease activity for the HCV polyprotein. Efficacy is characterized by comparing one or more prototoxophores with each other or with the free form of the toxin moiety included in the prototoxophore. Latency is characterized by the ability of the prototoxophore to remain in an inactive (non-toxic) form absent contact with the target viral enzyme, i.e., until activation takes place and the prototoxophore is converted to the toxophore form. Methods for determining the selectivity, potency, efficacy and latency of a candidate prototoxophore, i.e., an ECTA compound that has been designed based on the principles disclosed herein but not yet examined for the above identified criteria, are disclosed herein (Example 1) and additional methods for determining such parameters of a therapeutic agent are well known in the art.

The cytotoxic cancer chemotherapeutic drugs, mitoxantrone and amsacrine (FIG. 3) were selected as toxin moieties for preparing the exemplified NS3 protease prototoxophores. These compounds are DNA intercalating agents that induce DNA strand breaks, presumably by inhibiting topoisomerase II (see ref. 67, and references cited therein). Mitoxantrone is an approved agent for acute nonlymphocytic leukemia (ANLL) and has shown activity against solid tumors as a single modality agent. It is cytotoxic to both proliferating and nonproliferating cells, but has proven to be well tolerated. Amsacrine (m-AMSA) has significant antitumor activity in a wide range of animal models and has demonstrated clinical responses in ANLL, ovarian carcinomas, and lymphomas. The side effects, however, are more severe than with mitoxantrone.

Mitoxantrone and m-AMSA were selected for these studies based on their toxicity profiles, and because they can be modified with peptide or peptide-like moieties and still retain their cytotoxic properties (see FIG. 3; see, also, refs. 68 to 76). A result of catalytic activation of an NS3 prototoxophore is the presence of a small peptide, chemical linker, or the like following cleavage (see FIG. 4). Thus, where the activation of a prototoxophore by a viral enzyme results in a toxophore comprising a toxin and an additional peptide or other chemical group, the toxin is selected such that it maintains its potency in the presence of the additional group.

An advantage of using a DNA intercalating agent as the toxin moiety of an NS3 protease prototoxophore is that the negative charge required of the substrate domain for binding of the NS3 protease can contribute an electrostatic repulsion with respect to the phosphate backbone of DNA, thereby reducing or inhibiting the ability of the intercalating agent component of the prototoxophore to intercalate into cellular DNA (see Example 1A). These characteristics contribute to the relatively non-toxic activity of the prototoxophore, and result in the production of a latent cytotoxic agent that remains inactive, and relatively non-toxic, until it is activated by NS3 protease cleavage. Thus, by selecting a toxin moiety based, in part, on the characteristics of the substrate domain and target enzyme, and in view of the mechanism by which the toxin mediates its toxic effect, particularly useful prototoxophores can be designed, including those have desirable selectivity, potency, efficacy and latency.

In order to achieve reasonable hydrolysis rates with an NS3 protease, the NS3 protease prototoxophore, like any NS3 substrate, requires an extended binding interaction that spans from the non-prime side to the prime side (typically P6 to P4′) within NS3 (see ref. 66; see, also, ref. 117). The peptide sequences used in the exemplified NS3 protease prototoxophores (Table III) were based upon a consensus sequence, (Glu/Asp)-Xaa-Val-Val-(Leu/Pro)-Cys-(Ser/Ala) (SEQ ID NO: 21; scissile bond between Cys and Ser or Ala; Xaa indicates any amino acid) that is more active than the natural cleavage sites within the viral polyprotein, and on sequences that were shown to be resistant to proteolysis by host (yeast) proteases (see ref. 79). The toxin moiety in the exemplified prototoxophores was a 1,4-diamino anthraquinone (see FIG. 3), which is related to mitoxantrone. This chromophore is about 100-fold less potent than mitoxantrone, and is readily available and easily synthesized.

TABLE III
IC₅₀ values (μM) of HCV ECTA compounds.

| | 5759 | 5761 | 5763 | 5765 | 5766 | 5767 | 5768 |
|---|---|---|---|---|---|---|---|
| CCD18co | 17.3+/−4.4 | 24.4 | | 28 | 10.2 | 13.2 | 32 |
| | | | | 33 | | | |
| | | | | 38 | | | |
| Det551 | 7.6+/−1.2 | 17.5 | 17 | 15 | 5.3 | 5.1 | |
| | | 40 | | 16 | | 8 | |
| | | 16 | | | | | |
| MCF7TDX | 7.3+/−1.3 | 34.6 | 6 | 7 | 8.5 | 8 | 14 |
| | | 5 | | 8 | | | |
| | | | | 15 | | | |
| HepG2 | 13+/−1.9 | 51.1 | | 18 | 24.5 | 13.8 | 24 |
| | | | | 21 | | | |
| Hep3B | 12.4 | | | 25 | | | |
| | 21.7 | | | 26 | | | |

IC₅₀ values of various potential HCV ECTA compounds (5761-5768) were determined using CCD 18co (normal colon epithelial cells), Det551 (skin fibroblasts), MCF7TDX (Tomudex resistant breast carcinoma cell line), HepG2 and Hep3B (hepatocellular carcinoma cell lines). Compound 5759 mimics the “released toxin” and serves as a positive control. IC₅₀=the concentration of HCV ECTA compound that inhibits cellular growth by 50%.

The NS3 protease prototoxophores and synthetic toxophore forms thereof (Table I) were examined for cytotoxicity and DNA intercalating activity. As disclosed herein, several of the prototoxophores were substantially less toxic than the activated toxophore form, and the activated toxophore forms preferentially intercalated into DNA as compared to the prototoxophores (Example 1A). Furthermore, cytotoxicity correlated with DNA intercalating ability (Example 1C), thus allowing the identification of NS3 protease prototoxophores that can be useful for reducing or inhibiting viral infectivity.

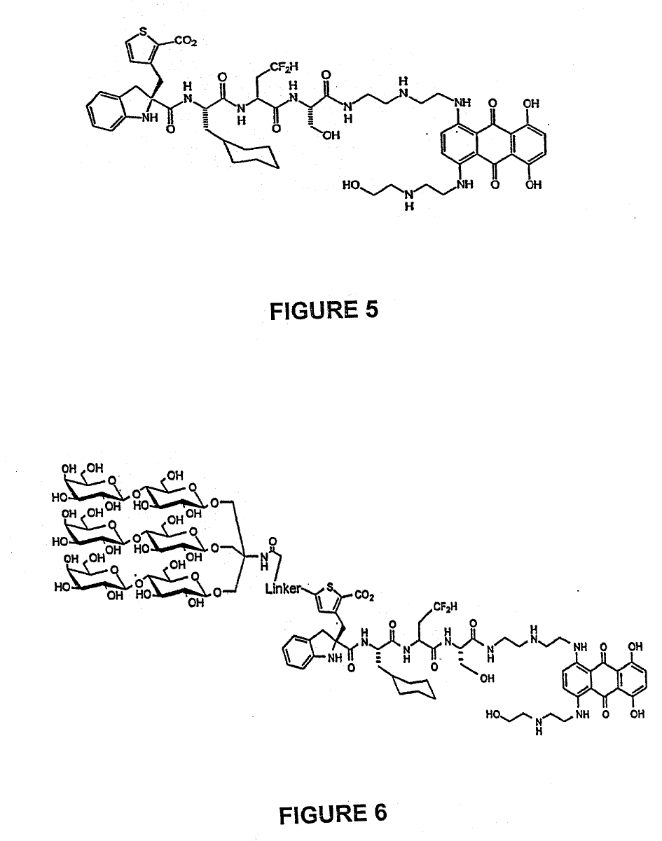

Although the exemplified prototoxophores are peptidic in nature (Table III), the substrate domain, including the recognition subdomain or modification domain or both, can be composed of peptides, peptidomimetics, polynucleotides or combinations thereof. In addition, where the targeted viral enzyme is a protease or other enzyme that cleaves a substrate, such molecules, and including simple organic linkers, can be substituted for the portion of the cleavage product comprising the toxin moiety, for example, for the nonapeptide illustrated in FIG. 4. For example, FIG. 5 provides an example of an NS3 protease generated toxophore having a peptidomimetic substituted for the tripeptide shown in FIG. 4.

A mimetic of a peptide substrate domain for a viral enzyme can be identified, for example, by screening a database that contains libraries of potential mimics. For example, the Cambridge Structural Database contains a collection of greater than 300,000 compounds that have known crystal structures (ref. 118). This structural depository is continually updated as new crystal structures are determined and can be screened for compounds having suitable shapes, for example, the same shape as a substrate domain of a viral enzyme, as well as potential geometrical and chemical complementarity to a substrate domain bound by a viral enzyme. Where no crystal structure of a viral enzyme or its substrate is available, a structure can be generated using a computer program such as CONCORD (ref. 119). Another database, the Available Chemicals Director (Molecular Design Limited, Informations Systems; San Leandro Calif.), contains about 100,000 compounds that are commercially available and also can be searched to identify potential mimics of a substrate domain or portion thereof useful in a prototoxophore of the invention.

In addition, a mimic of a naturally occurring substrate domain specific for a viral enzyme, including a peptidomimetic, peptoid, polynucleotide, or the like can be prepared either as a combinatorial library of randomly generated molecules or based on varying a domain already identified as having the desired characteristics of a substrate domain. In addition, a peptide domain previously identified as having the characteristics of a substrate domain can be varied using combinatorial chemistry methods, including methods of variegation (see, for example, U.S. Pat. No. 5,837,500).

Methods for preparing combinatorial libraries of molecules are well known in the art and include, for example, methods of making a phage display library of peptides, which can be constrained peptides (see, for example, U.S. Pat. No. 5,622,699; U.S. Pat. No. 5,206,347; refs. 120, 121); a peptide library (U.S. Pat. No. 5,264,563); a peptidomimetic library (ref. 122); a nucleic acid library (refs. 122, 123, 124); an oligosaccharide library (refs. 126, 127, 128); a lipoprotein library (ref. 129); a glycoprotein or glycolipid library (ref. 130); or a chemical library containing, for example, drugs or other pharmaceutical agents (refs. 4 to 15, 131, 132).

Peptidomimetics can be particularly useful for preparing all or a portion of a substrate domain or other domain of a prototoxophore because they generally are not susceptible to degradation by proteases, nucleases, and the like, which a prototoxophore may be exposed to if administered to an individual. Similarly, peptidomimetics can be more resistant than peptides to degradation by acid or alkaline conditions that a prototoxophore may be exposed to, for example, upon oral administration to an individual. It is recognized, however, that peptides can be readily modified, for example, by substituting D-amino acids for naturally occurring L-amino acids, by modifying one or more amino acid side chains, or by modifying one or more bonds linking the amino acid residues, provided the modified bond or modified amino acid does not interfere with the ability of the viral enzyme to effect its catalytic activity, for example, with the ability of a target viral protease to cleave the prototoxophore to generate the active toxophore. Such modified peptides are contemplated within the present invention and provide advantages similar to those of peptidomimetics.

Polynucleotides also can be useful for constructing a substrate domain, or portion thereof, of a prototoxophore because polynucleotides can be conveniently synthesized and can be varied using relatively simple methods. In addition, polynucleotides are known to be able to specifically bind polypeptides, including enzymes such as transcription factors, nucleases, and the like, and synthetic molecules having such specificity are readily prepared and identified (see, for example, U.S. Pat. No. 5,750,342; refs. 123-125).

The term “polynucleotide” is used broadly herein to mean a sequence of two or more deoxyribonucleotides or ribonucleotides that are linked together by a phosphodiester bond. As such, the term “polynucleotide” includes RNA and DNA, which can be a gene or a portion thereof, a cDNA, a synthetic polydeoxyribonucleic acid sequence, or the like, and can be single stranded or double stranded, as well as a DNA/RNA hybrid. Furthermore, the term “polynucleotide” as used herein includes naturally occurring nucleic acid molecules, which can be isolated from a cell, as well as synthetic molecules, which can be prepared, for example, by methods of chemical synthesis or by enzymatic methods such as by the polymerase chain reaction (PCR). In various embodiments, a polynucleotide of the invention can contain nucleoside or nucleotide analogs, or a backbone bond other than a phosphodiester bond (see above).

In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. However, a polynucleotide also can contain nucleotide analogs, including non-naturally occurring synthetic nucleotides or modified naturally occurring nucleotides such that a polynucleotide containing the synthetic or modified nucleotides have desirable characteristics such as resistance to nucleases. Such nucleotide analogs are well known in the art and commercially available, as are polynucleotides containing such nucleotide analogs (see refs. 133 to 135).

The covalent bond linking the nucleotides of a polynucleotide generally is a phosphodiester bond. However, the covalent bond also can be any of numerous other bonds, including a thiodiester bond, a phosphorothioate bond, a peptide-like bond or any other bond known to those in the art as useful for linking nucleotides to produce synthetic polynucleotides (see, for example, refs. 136, 137). The incorporation of non-naturally occurring nucleotide analogs or bonds linking the nucleotides or analogs can be particularly useful where the polynucleotide is to be exposed to an environment that can contain a nucleolytic activity, including, for example, a tissue culture medium or upon administration to a living subject, since the modified polynucleotides can be less susceptible to degradation. Furthermore, the incorporation of a peptide-like bond in a polynucleotide that mimics a substrate domain for a viral protease can allow the protease to cleave the polynucleotide substrate domain, similarly to as if the substrate domain was a peptide.

A polynucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template (ref. 134).

In view of the characteristics of the prototoxophores exemplified herein and the ability to construct prototoxophores having desirable characteristics, it will be recognized that the prototoxophores of the invention are useful for treating, preventing or otherwise reducing the spread of a viral infection. Accordingly, the present invention provides a method of reducing or inhibiting viral infectivity. As used herein, the term “viral infectivity” refers to the ability of a virus to infect a cell or to be transmitted from one cell to another. Thus, reference to “reducing or inhibiting” viral infectivity indicates that the ability of a virus to infect a cell or to be transmitted from one cell to another is less, due to the practice of a method of the invention, than it would be absent such practice. It should be recognized that the terms “reducing” and “inhibiting” are used together because, in some cases, it may be difficult to establish whether viral infectivity is completely inhibited or is reduced below the level of detection of a particular assay used to monitor the viral infectivity.

In general, a method of the invention reduces or inhibits viral infectivity by killing or decreasing the viability of the cells that are infected by the virus or by reducing or inhibiting the production of infectious virions by the infecting virus or the virally infected cell. As such, a method of the invention can affect the infecting virus or the virally infected target cell such that, upon conversion of a prototoxophore to a toxophore, the production of infectious virions is reduced or inhibited or the viability of the target cell is decreased, thereby reducing the likelihood that the infecting virus can lead to further infection of other cells. As used herein, the term “decreasing the viability,” when used in reference to the effect of a toxophore on a target cell, means that the target cell has been rendered less able to compete with a corresponding uninfected cell that has not been exposed to the toxophore. As such, the term “decreasing the viability” encompasses, for example, cells that are rendered incapable of mitosis, where the corresponding normal cells typically traverse the cell cycle; cells that are rendered defective in a metabolic pathway, where the pathway is typically active in corresponding normal cells; cells in which a pathway leading to cell death such as an apoptotic pathway has been induced, where the pathway is inactive in corresponding normal cells; and the like. As a result of the decrease in viability, the ability of an infecting virus to further infect other cells is reduced or inhibited.

A method of reducing or inhibiting viral infectivity can be performed by contacting a cell that is infected with a virus or is susceptible to infection with a virus with an amount of a synthetic prototoxophore that, when converted in a cell by a viral enzyme to a toxophore, is sufficient to decrease the viability of the cell containing the viral enzyme or to reduce or inhibit the production of infectious virions in or by the virally infected cell. As used herein, the term “contacting,” when used in reference to a prototoxophore and a cell, means that the prototoxophore is placed in sufficient proximity to the cell such that it can associate with the cell or a molecule expressed on the surface of the cell. As such, the term “contacting” has its commonly understood meaning, with the caveat that the prototoxophore need not be initially directly contacted with the cell, but can, for example, diffuse to the cell through a cell culture medium, or circulate to the cell through the vascular system, or the like.

The cell to be contacted with the prototoxophore, and which can be infected with a virus, can be any cell, including, for example, a cell of a cell line that has been adapted to long term continuous culture, in which case the cells generally are contacted with the prototoxophore in cell culture or following administration to a host animal (see Example 1), or cells of an individual that have been removed or isolated from the individual and contacted with the prototoxophore ex vivo, or that are contacted in situ in the individual or in a portion of the individual isolated, for example, by a shunt. Furthermore, the cell can be a cell of any eukaryotic organism that is susceptible to a viral infection, and generally is a vertebrate cell, particularly a mammalian cell, including a human cell such as a lymphocyte, a nerve cell, connective tissue cell, a muscle cell, an epithelial cell, a neuronal cell, a hepatocyte, and the like. As disclosed herein, the prototoxophore can include a targeting domain, which facilitates the likelihood that the prototoxophore will interact with a viral enzyme if present in a cell.

The present invention also provides a method of ameliorating the severity of a viral infection in an individual. Such a method can be performed by administering to the individual having or susceptible to a viral infection an amount of a synthetic prototoxophore that, when converted by a viral enzyme to a toxophore in a cell in the individual, decreased the viability of the cell. The viral infection can be any viral infection, including a human viral infection, and the virus can be any virus, particularly a virus that causes or exacerbates a disease. Thus, the infection can be, for example, a human immunodeficiency virus (HIV) infection such as with HIV-1, a herpes simplex virus (HSV) infection such as with HSV-1 or HSV-2, a rhinovirus infection, a hepatitis virus infection such as with hepatitis A, B or C virus, a picomavirus infection, a cytomegalovirus infection, or the like, provided the virus expresses a viral enzyme that can be targeted by a prototoxophore of the invention.

As used herein, the term “ameliorating,” when used in reference to the severity of a viral infection, means that the clinical signs or the symptoms associated with the viral infection are lessened. In general, amelioration of the severity of a viral infection can be identified by performing assays that directly monitor the presence of the virus, including, for example, antibody based assays, histopathological examination of an infected tissue or organism, electron microscopic examination, viral infectivity assays using standardized conditions, and the like. In addition, the skilled clinician will recognize that the evaluation of the severity of a viral infection will depend on the particular viral infection, and will know appropriate clinical parameters to examine. For example, where the viral infection is an HCV infection, it will be recognized that the presence or level of HCV RNA in a liver biopsy sample as determined using a reverse transcriptase-polymerase chain reaction (RT-PCR) based assay can be prognostic of the severity of the HCV infection and can be used to identify amelioration of the infection due to administration of a prototoxophore. Similarly, where the viral infection is HIV infection, it will be recognized that HIV viral load as determined by titrating HIV particles present in a blood sample can be indicative of the severity of HIV infection and can be used to identify amelioration of an HIV infection due to administration of a prototoxophore. Where the viral infection is a rhinovirus or influenza virus infection, i.e., the individual is suffering from a cold or the flu, an indication by the individual that he or she ‘feels better’ also can be indicative that the severity of the viral infection is ameliorated.

A method of the invention can be performed by administering the prototoxophore as a single treatment, either as a bolus injection or by infusion over a period of time, or can be performed as a series of separate treatments over a period of time, including days, weeks, or longer. An amount of a prototoxophore that, when converted to a toxophore, is sufficient to allow the selective killing of viral infected cells in a subject can be determined using the methods disclosed herein, and can further be optimized using routine clinical methods, including Phase I, II and III clinical trials. Efficacy of the method similarly can be determined using routine clinical methods, which, as discussed above, will depend in part on the nature of the infecting virus.

A prototoxophore generally is administered to an individual as a composition that includes a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the prototoxophore. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. It will be recognize that the choice of a pharmaceutically acceptable carrier, including a physiologically acceptable compound, depends, for example, on the physico-chemical characteristics of the prototoxophore and on the route of administration of the composition, which can be, for example, orally or parenterally such as intravenously, and by injection, intubation, or other such method known in the art. The pharmaceutical composition also can contain a second agent, for example, an additional therapeutic modality that can contribute to treatment of the primary viral infection or secondary effects caused by the infection; can be an antibiotic or antifungal agent, which can treat or prevent an opportunistic infection by a bacterium, fungus, protozoan, or other microbial organisms that the virally infected individual may also be infected with or susceptible to infection; can be a cancer chemotherapeutic agent where the individual is suffering from a cancer; or any other therapeutic modality that does not adversely affect the ability of the prototoxophore to effect its action. Additionally, one can enhance the therapeutic efficacy of a second known or yet to be discovered therapeutic by administering an effective amount of a compound of this invention. Examples of “second therapeutics” include one or more compounds disclosed in U.S. Pat. No. 6,245,750 and International Application No. PCT/US00/20008 or a compound having the structure

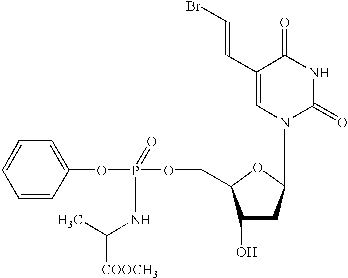

Administration can be prior to, subsequent to or concurrently with the other therapy.

The prototoxophore can be administered as a free compound, or can be incorporated within an encapsulating material such as into an oil-in-water emulsion, a microemulsion, micelle, mixed micelle, liposome, microsphere or other polymer matrix (see, for example, refs. 138, 139). Liposomes, for example, which consist of phospholipids or other lipids, are nontoxic, physiologically acceptable and metabolizable carriers that are relatively simple to make and administer. “Stealth” liposomes (see, for example, U.S. Pat. Nos. 5,882,679; 5,395,619; and 5,225,212) are an example of such encapsulating materials particularly useful for preparing a pharmaceutical composition useful for practicing a method of the invention, and other “masked” liposomes similarly can be used, such liposomes having the advantage of extending the time that the prototoxophore remains in the circulation, where desired. Cationic liposomes provide the further advantage that they can be modified with specific receptors or ligands, thereby allowing targeting of the prototoxophore (ref. 141).

The route of administration of a pharmaceutical composition containing the prototoxophore will depend, in part, on the chemical structure of the compound. Peptides and polynucleotides, for example, are not particularly useful when administered orally because they can be degraded in the digestive tract. However, as disclosed herein, methods for chemically modifying peptides, for example, to render them less susceptible to degradation by endogenous proteases or more absorbable through the alimentary tract are well known (see, for example, refs. 122, 132). In addition, a peptide agent can be prepared using D-amino acids, or can contain one or more domains based on peptidomimetics, which are organic molecules that mimic the structure of peptide domain; or based on a peptoid such as a vinylogous peptoid.

A pharmaceutical composition as disclosed herein can be administered to an individual by various routes including, for example, orally or parenterally, such as intravenously, intramuscularly, subcutaneously, intraorbitally, intracapsularly, intraperitoneally, intrarectally, intracisternally or by passive or facilitated absorption through the skin using, for example, a skin patch or transdermal iontophoresis, respectively. Furthermore, the pharmaceutical composition can be administered by injection, intubation, orally or topically, the latter of which can be passive, for example, by direct application of an ointment, or active, for example, using a nasal spray or inhalant, in which case one component of the composition is an appropriate propellant. Where the viral infection is localized in the individual, for example, in the liver, kidney, lungs, or other organ, the pharmaceutical composition containing the prototoxophore can be administered to the site of a viral infection, for example, intravenously or intra-arterially into a blood vessel supplying the infected tissue or organ, or by inhalation into the lungs.

The total amount of a prototoxophore to be administered in practicing a method of the invention can be administered to an individual as a single dose, either as a bolus or by infusion over a relatively short period of time, or can be administered using a fractionated treatment protocol, in which multiple doses are administered over a prolonged period of time. One skilled in the art would know that the amount of the pharmaceutical composition to treat a pathologic condition in a subject depends on many factors including the age and general health of the subject as well as the route of administration and the number of treatments to be administered. In view of these factors, the skilled artisan would adjust the particular dose as necessary.

The prototoxophore can be formulated for oral formulation, such as a tablet, or a solution or suspension form; or can comprise an admixture with an organic or inorganic carrier or excipient suitable for enteral or parenteral applications, and can be compounded, for example, with the usual non-toxic, pharmaceutically acceptable carriers for tablets, pellets, capsules, suppositories, solutions, emulsions, suspensions, or other form suitable for use. The carriers, in addition to those disclosed above, can include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening or coloring agents and perfumes can be used, for example a stabilizing dry agent such as triulose (see, for example, U.S. Pat. No. 5,314,695).

The following examples are intended to illustrate but not limit the invention.

### EXAMPLE 1

### Characterization OF Hepatitis C Virus NS3 Protease Activate Prototoxophores

This example provides methods for characterizing prototoxophores useful for reducing or inhibiting an infection by hepatitis C virus (HCV).

Normal human colon epithelial cells (CCD18co), skin fibroblasts (Det551) Hepatocellular carcinoma cell lines (Hep3B and HepG2), breast cancer cell line (MCF7) and fibrosarcoma cell line (HT1080) were all purchased from the American Type Culture Collection (ATCC; Rockville Md.). MCF7TDX, human breast carcinoma cells resistant to 2 μM Tomudex™ have been described (ref. 98). Cells were cultured at 37° C. in 95% humidified air/5% CO₂ in RPMI 1640 culture medium containing 10% fetal calf serum (Life Technologies) and penicillin/streptomycin/fungizone. MCF7TDX cells were maintained continuously in 2 μM Tomudex™. Normal cells were passaged a maximum of 15 times to avoid senescence.

Peptidyl HCV ECTA compounds were synthesized via fragment condensation and other standard solution phase peptide techniques (refs. 99 to 102). 1,4-diaminoanthraquinone was obtained from Aldrich.

A. DNA Binding Assay

Since the HCV NS3 protease prototoxophores require a negatively charged substrate domain, which is selectively bound by the NS3 protease, a toxin moiety was selected that intercalates with DNA, which also has a net negative charge in a cell due to the phosphate backbone. The prototoxophores were designed such that the negative charge of the substrate domain would contribute to inhibiting intercalation of the toxin moiety of the prototoxophore into DNA, and such that this inhibition would be relieved in the activated toxophore due to cleavage by the NS3 protease. The effectiveness of this design element was examined by titrating the 5763 candidate prototoxophore or the 5759 toxophore, which is the putative reaction product generated upon activation by HCV NS3 protease, with duplex DNA (see Table I).

Ten A₂₆₀ units (1 A₂₆₀ unit=50 Tg) of poly(dA-dT)/poly(dA-dT) (MW=2.6×10⁶, average length=4057 base pairs; Pharmacia Biotech) were reconstituted in 10 ml of 1X phosphate buffered saline (PBS) to produce a 20 nM duplex DNA stock solution (approximately 40 TM intercalator binding sites). The 5763 candidate prototoxophore or the 5759 toxophore was diluted to 10 TM or 12 TM final concentration in a quartz microcuvette in the absence or presence of 0.1, 0.3, 1, 3, 10, or 30 TM DNA (expressed in units of available intercalator binding sites). The UV/visible spectrum was measured from 700 to 200 nm (baseline corrected with 1X PBS blank) on a Cary 50 spectrophotometer.

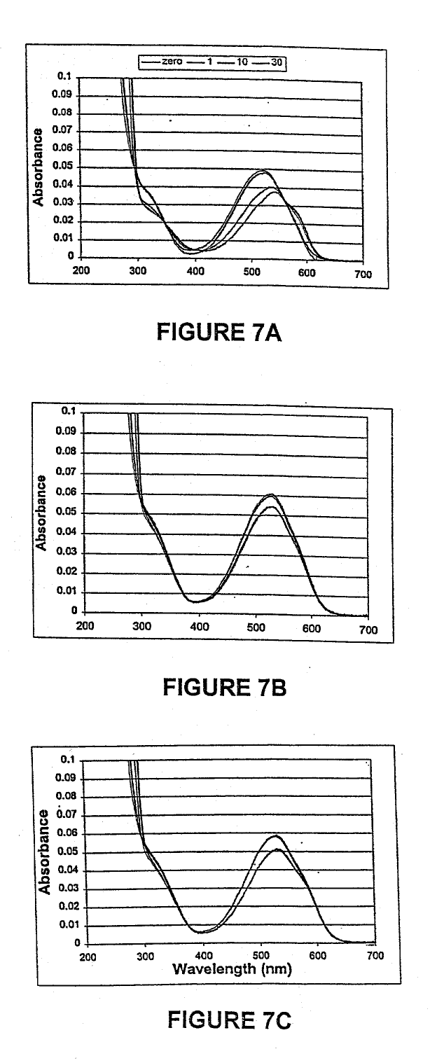

Intercalation into DNA by a chromophore generally results in a discernable shift in the UV/visible spectrum. Since the chromophores in the 5763 and 5759 compounds are identical, the direction and magnitude of the spectral shifts upon intercalation were expected to be the same, and any differences could be attributed to the quality and extent of intercalation, and can be used to infer binding affinity. The visible peak of the 5759 toxophore showed hypsochromic and bathochromic shifts upon addition of duplex DNA (FIG. 7A). Also a shoulder on the main peak appeared at 590 nm with the formation of a clear isosbestic point at 575 nm; this shoulder was not observed in the absence of DNA. In contrast, the spectrum of the 5763 candidate prototoxophore remained relatively unchanged at short incubation times (<60 min) displaying only a slight dimunition in absorptivity (FIG. 7B). However, upon prolonged incubation times (5 hr), a slight red shift became apparent and a shoulder started to develop at 590 nm (FIG. 7C).

These results indicate that the 5759 toxophore readily intercalated into duplex DNA, whereas the 5763 candidate prototoxophore had only a weak DNA binding interaction and began to intercalate only after a relatively long exposure to duplex DNA. These results also indicate that the 5759 toxophore has a much greater affinity for duplex DNA than the 5763 candidate prototoxophore.

B. HCV NS3 Protease Cleavage of Prototoxophores

Recombinant HCV NS3 protease is produced by transforming E. coli BL21(DE3) cells with pET28NS3/4A (see Section D, below) or with expression vectors harboring various combinations of the HCV NS3 and/or NS4 sequence. The HCV NS3/4A proteins are expressed in E. coli in response to IPTG induction, and purified by affinity chromatography on a Ni²⁺ His Bind Metal chelation resin (Novagen) according to the manufacturer's protocol. The purified enzyme is stored at −80° C.

Purified recombinant—NS3/NS4 fusion protein (SEQ ID NO:6), which includes amino acid residues 1029 to 1207 of HCV NS3 linked through a Gly-Ser-Gly-Ser (SEQ ID NO: 7) to amino acids 1678 to 1689 of HCV NS4A, is assayed for the ability to cleave the ECTA substrates as previously described (ref. 108). Briefly, candidate HSV NS3 protease prototoxophores are incubated with varying amounts of protease at 37° C. in 25 mM Tris-HCl (pH 7.5), 150 mM NaCl, 0.5 mM EDTA, 10 mM DTT, 10% glycerol, and 0.05% n-octyl- [symbol] -D-octyl glucoside ( [symbol] OG) for 15 min, then quenched with 1% TFA (0.1% final TFA). Reaction products (toxins) are quantitated by reverse-phase PLC (C-18). Peak areas are determined at 254 nm and the identity and amount of product verified by comparison with authentic control material.

This assay provides a means to confirm that a candidate HCV NS3 protease prototoxophore is a substrate for the protease and is cleaved to produce a putative activated toxophore.

C. Cytotoxicity Studies

The cytotoxic activity of various candidate prototoxophores was assessed by determining the IC₅₀ value (concentration that inhibits cellular growth by 50%) in cellular proliferation assays and compared with the results obtained using the 5759 toxophore. Exponentially growing cells were transferred at a density of 1000 to 5500 cells per well to a 96 well tissue culture plate and allowed to attach for 24 hr. HCV ECTA compounds were applied in duplicate half log serial dilutions. After an additional 72 hr incubation, surviving adherent cells were washed once with PBS and stained with 0.5% crystal violet in methanol. Sorenson's buffer (0.025 M sodium citrate, 0.025 M citric acid in 50% ethanol) was added to each well and absorbance at 540 nm was monitored (absorbance at 540 nm correlates with cell survival; see refs. 103 to 105). Semi-adherent or non-adherent cells were stained with CYQUANT stain according to the protocol provided with the cell proliferation assay kit (C-7026; Molecular Probes; Eugene Oreg.). Fluorescence (485 nM excitation, 535 nM emission) was monitored. IC₅₀ values were derived from sigmoid curves fit according to the Hill inhibitory Emax model (ref. 106).

Results from cytotoxicity assays using normal human cells (CCD18co and Det551) and human tumor cell lines (MCF7TDX, HepG2 and Hep3B) are shown in Table III. The cell lines examined are devoid of HCV NS3 protease activity and, therefore, the HCV NS3 protease prototoxophores were expected to be less toxic to these cells than the 5759 toxophore (see Table I), and, in fact, the 5761 and 5765 candidate prototoxophores had slightly higher IC₅₀ values (i.e., are less potent) with the two normal cell lines and with the HepG2 tumor cell line as compared to the IC₅₀ values of the 5759 toxophore (Table III).

Since only a slight difference in the IC₅₀ values was observed in the experiments described above, in which the cells were incubated for 72 hr with the HCV ECTA compound, additional cytotoxicity assays, in which the cells were exposed to the compounds for various periods of time, were performed. A clear difference was observed in the toxicity profile of the 5761 and 5763 candidate prototoxophores as compared to the the 5759 toxophore (Table IV). The 5759 toxophore was toxic to the cells after an exposure of 6 hr, whereas the 5761 and 5763 candidate prototoxophores showed little toxicity at this time point. In comparison, the 5767 candidate prototoxophore (Table I) showed a similar toxicity profile as the 5759 toxophore.

TABLE IV
IC₅₀ values (μM) of HCV ECTA Compounds after various times of
drug exposure

| | 5759 | | 5761 | | 5763 | | 5767 |
|---|---|---|---|---|---|---|---|
| | | MCF7/ | | MCF7/ | | MCF7/ | |
| | Det551 | TDX | Det551 | TDX | Det551 | TDX | Det551 |
| 2 h | 105, 50 | 17 | >150, | >150 | >150 | >150 | >150 |
| | | | >150 | | | | |
| 6 h | 17, 9 | 7 | >150, | >150 | 120 | >150 | 13 |
| | | | 126 | | | | |
| 12 h | 12, 9 | 6 | >150, | 89 | 84 | 116 | 11 |
| | | | 88 | | | | |
| 24 h | 9, 7 | 5 | 134, | 28 | 48 | 32 | 11 |
| | | | 50 | | | | |
| 72 h | 7, 4 | 4 | 40, | 5 | 17 | 7 | 8 |
| | | | 16 | | | | |

Det551 or MCF7TDX cells were treated with compounds 5759, 5761, 5763 and 5767 for 2, 6, 12, 24 and 72 hours. After the indicated times the compounds were removed, the cells were washed, incubated for a total of 72 hours and IC₅₀ values were determined. IC₅₀=the concentration of HCV ECTA compound that inhibits cellular growth by 50%.

These results indicate that the 5761 and 5763 candidate prototoxophores exhibit substantially less toxicity than the activated 5759 toxophore and, therefore, warrant further testing in paired cell lines with and without HCV NS3 protease activity. In addition, the results obtained with the 5763 candidate prototoxophore correlate with the decreased DNA affinity and intercalation observed with this compound (Section A, above). In comparison, the 5767 candidate prototoxophore was toxic in the absence of active HCV NS3 protease activity and, therefore, does not meet the criteria set for an HCV NS3 prototoxophore.

In order to confirm and extend these results, the 5762 and 5763 candidate prototoxophores were tested on CCD18co, Det551, MCF7TDX, HepG2 and Hep3B cells. After about a 15 hr period of drug treatment, the 5762 and 5763 candidate prototoxophores were less toxic to the cells than the 5759 toxophore (Table V). These results indicated the 5761, 5762 and 5763 candidate prototoxophores have characteristics required of an HCV ECTA therapeutic agent.

TABLE V
IC₅₀ values (μM) of HCV ECTA compounds on
various cell lines after 15 hour of drug exposure.

| | 5759 | 5762 | 5763 |
|---|---|---|---|
| CCD18co | 12 | 77 | 112 |
| Det551 | 8 | 46 | 131 |
| MCF7TDX | 8 | 132 | 146 |
| HepG2 | 14 | >150 | >150 |
| Hep3B | 22 | >150 | >150 |

Cells were treated with compounds 5759, 5762 or 5763 for 15 hours. After removal of the compounds, the cells were washed, incubated for a total of 72 hours and IC₅₀ values were determined. IC₅₀=the concentration of HCV ECTA compound that inhibits cellular growth by 50%.

D. Cell Based Assays for Conversion of Prototoxophore to Activated Toxophore

This example provides assays for assessing activation of a toxophore in cells that are transfected to stably express an HCV NS3 protease.

Expression plasmids encoding the HCV NS3 protease were constructed as follows. The Eco RI fragment encoding a fusion protein containing amino acid residues 1 to 151 of the human superoxide dismutase leader peptide and amino acid residues 946 to 1630 of the HCV polyprotein was isolated from the plasmid CF1SODP600 (ATCC) and cloned into the Eco RI site of pBluescriptSKII+plasmid (Stratagene; La Jolla Calif.) to yield pBlueSOD/HCV. The cDNA fragment encoding HCV protease NS3 and NS4A was generated using a two step PCR method (ref. 107).

In the first PCR step, the cDNA fragment encoding HCV protease NS3 amino acid residues 1027 to 1630 was amplified by PCR from plasmid pBlueSOD/HCV using the sense (forward) primer 5′-CAAGGATCCGCGCCCATCACGGCGTAC-3′(SEQ ID NO: 1; Bam HI site underlined) and the reverse primer 5′-GGGTGATCTCATTTTGAACAGCGCCCAGTCTGTATAG-3′(SEQ ID NO: 2). In addition, the cDNA fragment encoding HCV protease NS3 and NS4A (amino acid residues 1631 to 1711) was amplified from plasmid NS3NS4A using the sense primer 5′-CTATACAGACTGGGCGCTGTTCAAAATGAGATCACCC-3′(SEQ ID NO: 3) and the reverse primer 5′-CTGCTCGAGTTAGCACTCTTCCATTTCATC-3′(SEQ ID NO: 4; Xho I site underlined; engineered STOP codon in bold).

The two primary PCR products were gel purified and combined for the second PCR step, in which the complete NS3/NS4A sequence was amplified using the sense primer 5′-CAAGGATCCGCGCCCATCACGGCGTAC-3′(SEQ ID NO: 1) and the reverse primer 5′-CTGCTCGAGTTAGCACTCTTCCATTTCATC-3′(SEQ ID NO: 4). The amplified product (SEQ ID NO:5) was cloned into the Bam HI/Xho I sites of the pBluescriptSKII+plasmid to yield pBlueNS3/4A. The complete NS3/4A sequence was verified by sequencing. In SEQ ID NO:5, the first six nucleotides provide a Bam HI site and the last six nucleotides provide an Xho I site. The “taa” sequence preceding the Xho I site is a STOP codon. SEQ ID NO:5 encodes amino acids 1027 to 1711 of the HCV NS3/SN4A polypeptide.

The Barn HI/Xho I NS3/4A fragment was isolated from pBlueNS3/4A and cloned into the Barn HI/Xho I sites of the pCMV-Tag2b plasmid, which contains the FLAG epitope (Stratagene), and into the Bam HI/Xho I sites of the pET28a plasmid, which contains a His tag (Novagen), to yield pCMV-TagNS3/4A and pET28-NS3/4A, respectively. Constructs were sequenced to verify the in frame fusion of NS3 with the Flag epitope and His tag, respectively.

1. Identification of Transfected Cells Expressing NS3 Protease

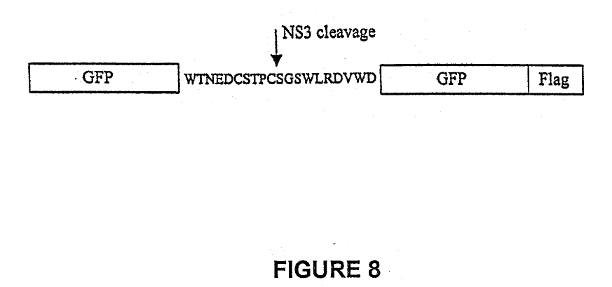

Hepatocellular carcinoma (Hep3B and HepG2), fibrosarcoma (HT1080), breast carcinoma (MCF7) and other tumor cells are transfected with the expression construct pCMV-TagNS3/4A or with the empty vector pCMV-Tag2b as a control. G418 resistant clones are isolated and screened for HCV NS3/4A gene expression by western blot analysis using the anti-Flag monoclonal antibody (Stratagene). In order to determine whether the expressed HCV NS3 protease has proteolytic activity, a mammalian expression vector is generated that expresses a fusion protein consisting of two green fluorescent proteins (GFP) separated by the HCV NS4B/5A cleavage site and Flag tagged on the carboxy-terminus (FIG. 8). A fused HCV NS4B/5A cleavage site is recognized and cleaved in trans by HCV NS3 protease (ref. 79).

The GFP-NS4B/5A mammalian expression plasmid is used to transiently transfect cells that overexpress HCV NS3 protease, and their corresponding controls that are devoid of NS3 protease activity. Transfection efficiency is monitored using a fluorescence microscope to determine the percentage of green cells, and cell lysates are prepared when the transfection efficiency is at least 50%. Western blot analysis is performed using the cell lysates and anti-GFP and anti-Flag antibodies. In cells devoid of NS3 protease activity, the identification of a protein having an apparent molecular mass of about 60 kDa, using the anti-GFP and anti-Flag antibodies, indicates that the fusion protein has not been cleaved. In comparison, identification of proteins having an apparent molecular mass of about 29 kDa, using the anti-GFP and anti-Flag antibodies, indicates that the fusion protein is cleaved and that the cells harbor HCV NS3 protease activity. This system allows for the identification of cells that express HCV NS3 protease having proteolytic activity.

2. Toxophore Activation by HCV NS3 Protease in a Cell

Candidate HCV NS3 protease prototoxophores are screened for activation and specificity using cell lines transfected to stably express the NS3 protease. Human hepatocellular carcinoma Hep3B or HepG2 cells, fibrosarcoma HT1080 cells or other tumor cells are transfected and cell lines stably expressing the HCV NS3 protease and NS4A cofactor. Cell lines containing the empty vector are selected and used as negative controls.

The transfected cells are contacted with the prototoxophore, and cytotoxicity of the cells is monitored. A greater amount of killing of cells that express the NS3 protease as compared to those lacking the protease indicates that the prototoxophore has entered the cells and has been converted to the activated toxophore. This approach was also used to discover active ECTA compounds targeting thymidylate synthase and α-lactamase (ref. 1; U.S. Pat. No. 6,159,706).

E. In Vivo Assay of Prototoxophore Efficacy

Mouse xenograft models are used to confirm the efficacy of an HCV NS3 protease prototoxophore in vivo. Hepatocellular carcinoma Hep3B cells are transfected to stably express the HCVNS3/NS4 proteins (see Section D, above), then the tumor cells are injected in the mid-back region of 4 to 6 week old athymic mice. After the tumor is established, mice are randomized and divided in different treatment groups. Efficacy of the HCV ECTA compounds are monitored by determining the difference in average tumor size between mice treated with a candidate HCV NS3 protease prototoxophore and those treated with vehicle, only (control). These stable transfection experiments also can be performed using HT1080 cells, MCF7 cells, or any other tumor cell line that is tumorigenic in an experimental animal.

Toxicity of the prototoxophore is examined and doses below that which causes substantial toxicity are selected. In vivo specificity of the prototoxophore for HCV NS3/4A expressing cells is assessed by comparing the efficacy of the prototoxophore against tumors induced by Hep3B cells harboring the empty vector with the efficacy against Hep3B tumors expressing the HCV NS3/4A. Efficacy is determined by comparing the average tumor sizes among the groups of mice. An efficacious HCV NS3 protease prototoxophore inhibits the growth or causes a regression in the size of HCV NS3 protease positive tumors, but not of the control NS3 protease negative tumors. Efficacy can also be determined by monitoring the activity, appetite, or survival of the mice in the various groups.

### References

(1) Lackey, D. B. et al. (2001) Biochem Pharmacol 61:179-189.

(2) Krausslich, H. G. and Wimmer, E. (1998) Annu Rev Biochem 57:701-754.

(3) Patick, A. K. and Potts, K. E. (1998) Clin Microbiol Rev 11:614-627.

(4) Amidon, G. L. and Lee, H. J. (1994) Annu Rev Pharmacol Toxicol 34:321-341.

(5) Eichler, J. et al. (1995) Med Res Rev 15:481-496.

(6) Al-Obeidi, F. et al. (1998) Mol Biotechnol 9:205-223.

(7) Ripka, A. S. and Rich, D. H. (1998) Curr Opin Chem Biol 2:441-452.

(8) Smith, A. B. et al. (1996) Bioorg Med Chem 4:1021-1034.

(9) Dragovich, P. S. et al. (1993) J Med Chem 42:1203-1212.

(10) Dorner, B. et al. (1996) Bioorg Med Chem 4:709-715.

(11) Dooley, C. T. and Houghten, R. A. (1999) Biopolymers 51:379-390.

(12) Hruby, V. J. et al. (1997) Biopolymers 43:219-266.

(13) Hruby, V. J. and Balse, P. M. (2000) Curr Med Chem 7:945-970.

(14) Hruby, V. J. et al. (1997) Curr Opin Chem Biol 1: 1 14-119.

(15) Ho, W. B. and Broka, C. (2000) J Org Chem 65:6743-6748.

(16) Gulnik, S. et al. (2000) Vitam Horm 58:213-256.

(17) Swanstrom, R. and Erona, J. (2000) Pharmacol Ther 86:145-170.

(18) Erickson, J. W. et al. (1999) AIDS 13:S189-204.

(19) Louis, J. M. et al. (2000) Adv Pharmacol 49:111-146.

(20) Pawlotsky, J. M. (2000) Hepatology 32:889-896.

(21) Polyak, S. J. and Gerotto, M. (2000) Forum (Genova) 10:46-58.

(22) Taylor, D. R. et al. (2000) Microbes Infect 2:1743-1756.

(23) Wright, M. et al. (2000) J Infect 41:1-4.

(24) Brillanti, S. et al. (2000) Hepatology 32:630-634.

(25) Sarisky, R. T. et al. (2001) J Virol 75:1761-1769.

(26) Pramod, N. P. et al. (2000) Microbiol Immunol 44:241-247.

(27) Lipsitch, M. et al. (2000) Antimicrob Agents Chemother 44:2824-2835.

(28) Chakrabarti, S. et al. (2000) J Infect Dis 181:2055-2058.

(29) Pillay, D. (1998) Commun Dis Public Health 1:5-13.

(30) Hodinka, R. L. (1997) Infect Dis Clin North Am 11:945-967.

(31) Monto, A. S. and Arden, N. H. (1992) Clin Infect Dis 15:362-367; discussion 368-369.

(32) Cuthbert, J. A. (1994) Clinical Microbiology Reviews 7:505-532.

(33) Alter, M. J. (1997) Hepatology 26:62S-65S.

(34) Cohen, J. (1999) Science 285:26-30.

(35) Kuo, G. et al. (1989) Science 244:362-364.

(36) Choo, Q. L. et al. (1989) Science 244:359-362.

(37) Davis, G. L. et al. (1998) New England Journal of Medicine 339:1493-1499.

(38) Fields, B. N. et al. (1996) In Fields Virology; Third ed.; Lippincott-Raven Publishers: Philadelphia.

(39) Bartenschlager, R. (1997) Intervirology 40:378-393.

(40) Hussy, P. et al. (1996) Virology 224:93-104.

(41) Lin, C. et al (1994) J Virol 68:5063-5073.

(42) Mizushima, H. et al. (1994) J Virol 68:6215-6222.

(43) Mizushima, H. et al. (1994) J Virol 68:2731-2734.

(44) Reed, K. E. et al. (1995) J Virol 69:4127-4136.

(45) Hirowatari, Y. et al. (1993) Arch Virol 133:349-356.

(46) Hijikata, M. et al. (1993) J Virol 67:4665-4675.

(47) Grakoui, A. et al. (1993) Proc Natl Acad Sci USA 90:10583-10587.

(48) Tomei, L. et al. (1993) J Virol 67:4017-4026.

(49) Dymock, B. W. et al. (2000) Antivir Chem Chemother 11:79-96.

(50) Ahmed, A. and Keeffe, E. B. (1999) J Gastroenterol Hepatol 14:S12-18.

(51) Manabe, S. et al. (1994) Virology 198:636-644.

(52) Grakoui, A. et al. (1993) J Virol 67:2832-2843.

(53) Eckart, M. R. et al. (1993) Biochem Biophys Res Commun 192:399-406.

(54) Kim, D. W. et al. (1995) Biochem Biophys Res Commun 215:160-166.

(55) Hahm, B. et al. (1995) J Virol 69:2534-2539.

(56) Failla, C. et al. (1994) J Virol 68:3753-3760.

(57) Urbani, A. et al. (1997) J Biol Chem 272:9204-9209.

(58) Kim, J. L. et al. (1996) Cell 87:343-355.

(59) Love, R. A. et al. (1996) Cell 87:331-342.

(60) Lechmann, M. and Liang, T. J. (2000) Semin Liver Dis 20:211-226.

(61) Heile, J. M. et al. (2000) J Virol 74:6885-6892.

(62) Forns, X. et al. (2000) Hepatology 32:618-625.

(63) Kamei, A. et al. (2000) Virology 273:120-126.

(64) Pancholi, P. et al. (2000) J Infect Dis 182:18-27.

(65) Lee, A. Y. et al. (2000) Vaccine 18:1962-1968.

(66) Ingallinella, P. et al. (1998) Biochemistry 37:8906-8914.

(67) Dorr, R. T. and vonHoff, D. D. (1994) Cancer Chemotherapy Handbook; 2 ed.; McGraw-Hill, pp 1020.

(68) Bourdouxhe, C. et al. (1995)Anticancer Drug Des 10:131-154.

(69) Bailly, F. et al. (1992) Anticancer Drug Des 7:83-100.

(70) Bailly, C et al. (1992) Biochem Pharmacol 43:457-466.

(71) Bailly, C. et al. (1996) Anticancer Drug Des 11:611-624.

(72) Boitte, N. et al. (1997) Anticancer Drug Des 12:481-501.

(73) Bourdouxhe-Housiaux, C. et al. (1996) Anticancer Drug Des 11:509-525.

(74) Bourdouxhe-Housiaux, C. et al. (1996) Biochemistry 35:4251-4264.

(75) Flock, S. et al. (1994) J Biomol Struct Dyn 11:881-900.

(76) Gatto, B. et al (1996) J. Med. Chem. 39:3114-3122.

(77) Qu, X. et al. (1999) Anticancer Drug Des 14:433-442.

(78) Arrowsmith, J. et al. (1999) Anticancer Drug Des 14:205-217.

(79) Kim, S. Y. et al. (2000) Anal Biochem 284:42-48.

(80) Pricer, W. E. and Ashwell, G. (1971) J Biol Chem 246:4825-4833.

(81) Ashwell, G. and Harford, J. (1982) Annu Rev Biochem 51:531-554.

(82) Spiess, M. (1990) Biochemistry 29:10009-10018.

(83) Schwartz, A. L. et al. (1982) J Biol Chem 257:4230-4237.

(84) Watanabe, Y. et al. (2000) J Biomater Sci Polym Ed 11:833-848.

(85) Lim, D. W. et al. (2000) Bioconjug Chem 11:688-695.

(86) Rosebrough, S. F. and Hartley, D. F. (2000) Cancer Biother Radiopharm 15:507-515.

(87) DeFeo-Jones, D. et al. (2000) Nat Med 6:1248-1252.

(88) Jones, G. B. et al. (2000) Bioorg Med Chem Lett 10:1987-1989.

(89) Khan, S. R. and Denmeade, S. R. (2000) Prostate 45:80-83.

(90) Hamstra, D. A. et al. (2000) Cancer Res 60:657-665.

(91) Chakravarty, P. K. et al. (1983) J Med Chem 26:638-644.

(92) Chakravarty, P. K. et al. (1983) J Med Chem 26:633-638.

(93) Carl, P. L. et al. (1980) Proc Natl Acad Sci USA 77:2224-2228.

(94) Connors, T. A. (1995) Stem Cells 13:501-511.

(95) Grove, J. I. et al. (1999) Anticancer Drug Des 14:461-472.

(96) Napier, M. P. et al. (2000) Clin Cancer Res 6:765-772.

(97) Bagshawe, K. D. et al. (1999) Curr Opin Immunol 11:579-583.

(98) Drake, J. C. et al. (1996) Biochem Pharmacol 51:1349-1355.

(99) Naithani, V. K. and Fohles, J. (1978) Hoppe Seylers Z Physiol Chem 359:1173-1181.

(100) Ondetti, M. A. et al. (1968) J Am Chem Soc 90:4711-4715.

(101) Nyfeler, R. (1994) Methods Mol Biol 35:303-316.

(102) Bodanszky, M. (1984) Principles of Peptide Synthesis; Springer-Verlag: Berlin, 307.

(103) Flick, D. A. and Gifford, G. E. (1984) J Immunol Methods 68:167-175.

(104) Gillies, R. J. et al. (1986) Anal Biochem 159:109-113.

(105) Reile, H. et al. (1990) Anal Biochem 187:262-267.

(106) Hill, A. (1913) Biochem J 7:471-480.

(107) Stark, M. R. and Johnson, A. D. (1994) Nature 371:429-432.

(108) Taremi, S. S. et al. (1998) Protein Sci 7:2143-2149.

(109) Basrur, et al. (2000) J. Biol. Chem. 275:14890-14897.

(110) Ferguson, et al. (2000) J. Org. Chem. 65:1218-1221.

(111) Chao, et al. (2000) J. Biol. Chem. 275:28345-28345

(112) Harrison's “Principles of Internal Medicine” 13th ed. (eds. Isselbacher et al., McGraw-Hill, Inc. 1994), pages 1830-1835.

(113) Schwarze, et al. (1999) Science 285:1569-1572.

(114) Derossi, et al. (1996) J. Biol. Chem. 271:18188.

(115) Hancock, et al. (1991), EMBO J. 10:4033-4039.

(116) Buss, et al. (1988) Mol. Cell. Biol. 8:3960-3963.

(117) Landro, et al. (1997), Biochemistry 36:9340-9348.

(118) Allen, et al. (1979) Acta Crystallogr. Section B, 35:2331.

(119) Rusinko, (1989) J. Chem. Int. Comput. Sci. 28:251.

(120) Scott and Smith, (1992) Science 249:386-390.

(121) Markland, et al. (1991) Gene 109:13-19.

(122) Blondelle, et al. (1995) Trends Anal. Chem. 14:83-92.

(123) O'Connell, et al. (1996) Proc. Natl. Acad. Sci., USA 93:5883-5887.

(124) Tuerk and Gold, (1990) Science 249:505-510.

(125) Gold, et al. (1995) Ann. Rev. Biochem. 64:763-797.

(126) York, et al. (1996) Carb. Res. 285:99-128.

(127) Liang, et al. (1996) Science 274:1520-1522.

(128) Ding, et al. (1995) Adv. Expt. Med. Biol. 376:261-269.

(129) de Kruif, et al. (1996) FEBS Lett. 399:232-236.

(130) Karaoglu, et al. (1995) J. Cell Biol. 130:567-577.

(131) Gordon, et al. (1994) J. Med. Chem. 37:1385-1401.

(132) Ecker and Crooke, (1995) BioTechnology 13:351-360.

(133) Lin, et al. (1994) Nucl. Acids Res. 22:5220-5234.

(134) Jellinek, et al. (1995) Biochemistry 34:11363-11372.

(135) Pagratis, et al. (1997) Nature Biotechnol. 15:68-73.

(136) Tam, et al. (1994) Nucl. Acids Res. 22:977-986.

(137) Ecker and Crooke, (1995) BioTechnology 13:351360.

(138) Gregoriadis, (1984) Liposome Technology, Vol. 1 (CRC Press, Boca Raton, Fla.

(139) Fraley, et al. (1981) Trends Biochem. Sci. 6:77.

(140) Morishita, et al. (1993) J. Clin. Invest. 91:2580-2585.

(141) Blondelle, et al. (1995) Trends Anal. Chem. 14:83-92.

(142) Ecker and Crooke, (1995) BioTechnology 13:351-360.

What is claimed is:

1. A synthetic viral prototoxophore, comprising a toxin moiety operatively incorporated into a substrate domain specific for a viral enzyme, bound and modified by said viral enzyme, thereby converting the prototoxophore to a toxophore.

2. The protoxophore of claim 1, wherein the substrate domain is a polypeptide.

3. The prototoxophore of claim 1, wherein the prototoxophore has the structure: 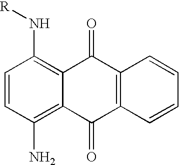
wherein R is (Glu/Asp)-Xaa-Val-Val-(Leu/Pro)-Cys-(Ser/Ala) wherein Xaa is any any amino acid. (Seq. ID No. 21) and wherein said compound may be in any enantiomeric, diasteriomeric, or stereoisomeric form, consisting of a D-form, L-form, α-anomeric form, and β-anomeric form or a pharmaceutically acceptable salt thereof.

4. The protoxophore of claim 1, wherein the comprises a polypeptide selected from the group consisting of the peptide sequences 5761 through 5771, as shown in Table 1.

5. The prototoxophore of claim 1, wherein the toxin moiety is 1,4, diaminoanthraquione.

6. The synthetic viral prototoxophore of claim 1, wherein said substrate at least is a single domain.

7. The synthetic viral protoxophore of claim 1, wherein said viral enzyme is a viral protease, which can bind to a first domain of the substrate domain and cleave a second domain and upon cleavage of said second domain, converts said prototoxophore to a toxophore.

8. The synthetic viral prototoxophore of claim 1, wherein said toxin moiety is a toxin, selected from the group consisting of an antimetabolite, an alkylating agent, a plant alkaloid and an antitumor antibiotic.

9. The synthetic viral prototoxophore of claim 1, wherein said toxin moiety is a DNA intercalating agent and said substrate domain comprises a substrate for a viral protease.

10. The synthetic viral protease of claim 9, wherein said viral protease is selected from the group consisting of a hepatitis virus protease, a human immunodeficiency virus protease, a rhinovirus protease, a herpes virus protease, an adenovirus protease, and a cytomegalovirus protease.

11. The synthetic viral prototoxophore of claim 9, wherein said viral protease is a hepatitis C virus (HCV) NS3 protease.

12. The synthetic viral prototoxophore of claim 11, wherein said HCV NS3 protease prototoxophores are selected from the group consisting of compounds 5761 to 5763 as set forth in Table I, and by compounds 5764 to 5766 and 5768 to 5771 as set forth in Table I.

13. A toxophore comprising the product of the structure of the product shown in FIG. 4, or compound 5759 or 5760 in Table I.

14. A method of reducing or inhibiting viral infectivity, comprising contacting a cell, which is infected with a virus or is susceptible to infection with a virus, with an effective amount of the synthetic viral prototoxophore of claim 1.

15. The method of claim 14, wherein said cells are cell lines adapted to long term continuous culture, cells isolated from a subject.

16. The method of claim 14, wherein said cell is a lymphocyte, nerve cell, connective tissue cell, muscle cell or a hepatocyte.

17. A method of ameliorating the severity of a viral infection in a subject, by administering to the subject an effective amount of said synthetic prototoxophore of claim 1.

18. The method of claim 17, wherein said vrus is selected from the group consisting of human immunodeficiency virus (HIV), a herpes simplex virus (HSV), a rhinovirus and a hepatitis virus.

19. The method of claim 17, further comprising administering to said subject an effective amount of an anti-viral agent.

20. A method for enhancing the anti-viral effect of an anti-viral agent comprising contacting a cell infected with a virus or is susceptible to infection with a virus with an effective amount of said anti-viral agent and an effective amount of the compound of claim 1.

21. The method of claim 20, wherein the anti-viral agent is a compound having the structure: 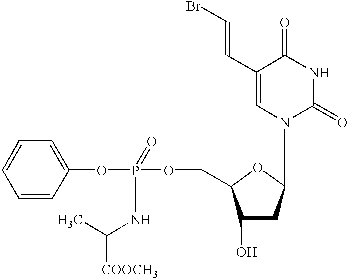
wherein said compound may be in any enantiomeric, diasteriomeric, or stereoisomeric form, consisting of a D-form, L-form, α-anomeric form, and β-anomeric form or a pharmaceutically acceptable salt thereof.

22. An assay to identify anti-viral agents, comprising contacting a virally infected cell with an amount of a candidate agent and comparing the ability of the candidate agent to inhibit the growth or infectivity of the virus in said virally infected cell to the ability of a compound of claim 1 to inhibit the growth or infectivity of the virus in said virally infected cell.
